SEQUENCE LISTING (34 sequences)

SEQ ID NO: 1 - protein, 9 aa, Artificial Sequence
  Synthetic polypeptide
IYNGKLFDL

SEQ ID NO: 2 - protein, 9 aa, Artificial Sequence
  Synthetic polypeptide
SSSQESVPK

SEQ ID NO: 3 - DNA, 819 bp, Artificial Sequence
  Synthetic polynucleotide
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtggatactggagccaatagtaagctgacatttggaaaaggaataactctgagtgttagaccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 4 - protein, 272 aa, Artificial Sequence
  Synthetic polypeptide
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVDTGANSKLTFGKGITLSVRPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 5 - protein, 113 aa, Artificial Sequence
  Synthetic polypeptide
KQEVTQIPAALSVPEGENLVLNCSFTDSAIYNLQWFRQDPGKGLTSLLLIQSSQREQTSGRLNASLDKSSGRSTLYIAASQPGDSATYLCAVDTGANSKLTFGKGITLSVRPD

SEQ ID NO: 6 - protein, 19 aa, Artificial Sequence
  Synthetic polypeptide
METLLGLLILWLQLQWVSS

SEQ ID NO: 7 - protein, 6 aa, Artificial Sequence
  Synthetic polypeptide
DSAIYN

SEQ ID NO: 8 - protein, 7 aa, Artificial Sequence
  Synthetic polypeptide
IQSSQRE

SEQ ID NO: 9 - protein, 11 aa, Artificial Sequence
  Synthetic polypeptide
AVDTGANSKLT

SEQ ID NO: 10 - DNA, 927 bp, Artificial Sequence
  Synthetic polynucleotide
atgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccaacctgcaaagcttgaggactcggccgtgtatctctgtgccagcagctatcatagctatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 11 - protein, 308 aa, Artificial Sequence
  Synthetic polypeptide
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSYHSYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 12 - protein, 112 aa, Artificial Sequence
  Synthetic polypeptide
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSYHSYGYTFGSGTRLTVV

SEQ ID NO: 13 - protein, 19 aa, Artificial Sequence
  Synthetic polypeptide
MGTRLLCWAALCLLGAELT

SEQ ID NO: 14 - protein, 5 aa, Artificial Sequence
  Synthetic polypeptide
SGHAT

SEQ ID NO: 15 - protein, 6 aa, Artificial Sequence
  Synthetic polypeptide
FQNNGV

SEQ ID NO: 16 - protein, 10 aa, Artificial Sequence
  Synthetic polypeptide
ASSYHSYGYT

SEQ ID NO: 17 - DNA, 828 bp, Artificial Sequence
  Synthetic polynucleotide
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttataggagcgcggttcggcagaagctgctctttgcaaggggaaccatgttaaaggtggatcttaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctaa

SEQ ID NO: 18 - protein, 275 aa, Artificial Sequence
  Synthetic polypeptide
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRSAVRQKLLFARGTMLKVDLNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 19 - protein, 115 aa, Artificial Sequence
  Synthetic polypeptide
AQTVTQSQPEMSVQEAETVTLSCTYDTSESDYYLFWYKQPPSRQMILVIRQEAYKQQNATENRFSVNFQKAAKSFSLKISDSQLGDAAMYFCAYRSAVRQKLLFARGTMLKVDLN

SEQ ID NO: 20 - protein, 20 aa, Artificial Sequence
  Synthetic polypeptide
MACPGFLWALVISTCLEFSM

SEQ ID NO: 21 - protein, 7 aa, Artificial Sequence
  Synthetic polypeptide
TSESDYY

SEQ ID NO: 22 - protein, 8 aa, Artificial Sequence
  Synthetic polypeptide
QEAYKQQN

SEQ ID NO: 23 - protein, 11 aa, Artificial Sequence
  Synthetic polypeptide
AYRSAVRQKLL

SEQ ID NO: 24 - DNA, 939 bp, Artificial Sequence
  Synthetic polynucleotide
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagctccccggggggccggtcctacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttctaa

SEQ ID NO: 25 - protein, 312 aa, Artificial Sequence
  Synthetic polypeptide
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSSPGGRSYNEQFFGPGTRLTVLEDLKNVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 26 - protein, 116 aa, Artificial Sequence
  Synthetic polypeptide
DTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSSPGGRSYNEQFFGPGTRLTVL

SEQ ID NO: 27 - protein, 19 aa, Artificial Sequence
  Synthetic polypeptide
MGTSLLCWMALCLLGADHA

SEQ ID NO: 28 - protein, 5 aa, Artificial Sequence
  Synthetic polypeptide
SEHNR

SEQ ID NO: 29 - protein, 6 aa, Artificial Sequence
  Synthetic polypeptide
FQNEAQ

SEQ ID NO: 30 - protein, 14 aa, Artificial Sequence
  Synthetic polypeptide
ASSSPGGRSYNEQF

SEQ ID NO: 31 - protein, 13 aa, Artificial Sequence
  Synthetic polypeptide
CAYRSAVRQKLLF

SEQ ID NO: 32 - protein, 16 aa, Artificial Sequence
  Synthetic polypeptide
CASSSPGGRSYNEQFF

SEQ ID NO: 33 - protein, 13 aa, Artificial Sequence
  Synthetic polypeptide
CAVDTGANSKLTF

SEQ ID NO: 34 - protein, 12 aa, Artificial Sequence
  Synthetic polypeptide
CASSYHSYGYTF

(19) United States
(12) Patent Application Publication
(10) Pub. No.: US 2024/0207406 A1
(43) Pub. Date: Jun. 27, 2024

(54) METHODS AND COMPOSITIONS COMPRISING MHC CLASS I PEPTIDES

(71) Applicant: BOARD OF REGENTS, THE UNIVERSITY OF TEXAS SYSTEM, Austin, TX (US)

(72) Inventors: Cassian YEE, Houston, TX (US); Ke PAN, Houston, TX (US); Yulun CHIU, Houston, TX (US)

(73) Assignee: BOARD OF REGENTS, THE UNIVERSITY OF TEXAS SYSTEM, Austin, TX (US)

(21) Appl. No.: 18/556,054

(22) PCT Filed: Apr. 18, 2022
(86) PCT No.: PCT/US2022/025223

§ 371 Date: Oct. 18, 2023

Related U.S. Application Data
(60) Provisional application No. 63/176,477, filed on Apr. 19, 2021.

(51) Int. Cl. A61K 39/00; A61K 45/06; C07K 7/06; C07K 14/725; C07K 14/74; C07K 16/32; C12N 5/0783; C12N 15/115; G01N 33/569

(52) U.S. Cl. CPC A61K 39/4644; A61K 39/0011; A61K 39/4611; A61K 39/4632; A61K 45/06; C07K 7/06; C07K 14/7051; C07K 14/70539; C07K 16/32; C12N 5/0636; C12N 15/115; G01N 33/56966; C07K 2317/31; C07K 2317/622; C07K 2318/00; C07K 2319/30; C07K 2319/32; C12N 2502/1121; C12N 2510/00

(57) ABSTRACT

The current disclosure fulfills a need in the art by providing methods and compositions for treating and vaccinating individuals against cancer. Accordingly, aspects of the disclosure relate to an isolated peptide comprising at least 70% sequence identity to a peptide of SEQ ID NO: 1 or 2. In some embodiments, the peptide comprises at least 6 contiguous amino acids of a peptide of SEQ ID NO:1 or 2. Further aspects relate to pharmaceutical compositions comprising the isolated peptide, nucleic acids encoding the peptide, and expression vectors and host cells comprising the nucleic acids of the disclosure. Also provided is an in vitro isolated dendritic cell comprising a peptide, nucleic acid, or expression vector of the disclosure.

This application claims priority of U.S. Provisional Patent Application No. 63/176,477, filed Apr. 19, 2021, which is hereby incorporated by reference in its entirety.

### BACKGROUND OF THE INVENTION

### I. Field of the Invention

This invention relates to the field of treatment of cancer.

### II. Background

Adoptive T cell therapy (ACT; also referred to as an “adoptive cell transfer”) has shown significant promise as a method for treating melanoma; unfortunately, this approach has also been hindered by limitations including toxicity towards non-cancerous tissues. ACT generally involves which involves infusing a large number of autologous activated tumor-specific T cells into a patient, e.g., to treat a cancer. ACT has resulted in therapeutic clinical responses in melanoma patients (Yee 2002; Dudley 2002; Yee 2014; Chapuis 2016). Generally, to develop effective anti-tumor T cell responses, the following three steps are normally required: priming and activating antigen-specific T cells, migrating activated T cells to tumor site, and recognizing and killing tumor by antigen-specific T cells. The choice of target antigen is important for induction of effective antigen-specific T cells.

While several tumor-associated antigens have been identified for melanoma and a handful of other solid tumor malignancies, there are few immunogenic targets for pancreatic, ovarian, gastric, lung, cervical, breast, and head and neck cancer. Identification and validation of novel epitopes and target antigens for these common and difficult to treat malignancies is warranted.

### SUMMARY OF THE INVENTION

The current disclosure fulfills a need in the art by providing methods and compositions for treating and vaccinating individuals against cancer. Accordingly, aspects of the disclosure relate to an isolated peptide comprising at least 70% sequence identity to a peptide of IYNGKLFDL (SEQ ID NO:1) or SSSQESVPK (SEQ ID NO:2). Also disclosed is a molecular complex comprising a peptide of the disclosure and a MHC polypeptide. Further aspects relate to pharmaceutical compositions comprising the isolated peptide, molecular complexes, nucleic acids encoding the peptide, expression vectors, and host cells comprising the nucleic acids and peptides of the disclosure. Also provided is an in vitro isolated dendritic cell comprising a peptide, nucleic acid, or expression vector of the disclosure. Also contemplated are polypeptides comprising one or more peptides of the disclosure.

Further aspects relate to a method of making a cell comprising transferring a nucleic acid or expression vector of the disclosure into a cell, such as a host cell. In some aspects, the method further comprises isolating the expressed peptide or polypeptide. Other aspects of the disclosure relate to a method of producing cancer-specific immune effector cells comprising: (a) obtaining a starting population of immune effector cells; and (b) contacting the starting population of immune effector cells with a peptide of the disclosure, thereby generating peptide-specific immune effector cells.

The disclosure also describes peptide-specific engineered T cells produced according to the methods of the disclosure and pharmaceutical compositions comprising the engineered T cells. Further aspects relate to a method of treating or preventing cancer in a subject, the method comprising administering an effective amount of a peptide, molecular complex, pharmaceutical composition, nucleic acid, expression vector, dendritic cell, peptide-specific T cells, polypeptide, TCR, or fusion protein of the disclosure to the subject. Also provided is a method of stimulating an immune response in a subject, the method comprising administering an effective amount of a peptide, molecular complex, pharmaceutical composition, nucleic acid, expression vector, dendritic cell, peptide-specific T cells, polypeptide, TCR, or fusion protein of the disclosure to the subject. Yet further aspects relate to a method of cloning a peptide-specific T cell receptor (TCR), the method comprising (a) obtaining a starting population of immune effector cells; (b) contacting the starting population of immune effector cells with the peptide of the disclosure, thereby generating peptide-specific immune effector cells; (c) purifying immune effector cells specific to the peptide, and (d) isolating a TCR sequence from the purified immune effector cells. Also provide is a method for prognosing a patient or for detecting T cell responses in a patient, the method comprising: contacting a biological sample from the patient with a peptide of the disclosure.

The disclosure also provides for a polypeptide comprising an antigen binding variable region comprising a CDR3 comprising the amino acid sequence of SEQ ID NO:9 or an amino acid sequence with at least 80% sequence identity to SEQ ID NO:9. Aspects relate to a polypeptide comprising an antigen binding variable region comprising a CDR3 comprising the amino acid sequence of SEQ ID NO:16 or an amino acid sequence with at least 80% sequence identity to SEQ ID NO:16. Also described is a polypeptide comprising an antigen binding variable region comprising a CDR3 comprising the amino acid sequence of SEQ ID NO:23 or an amino acid sequence with at least 80% sequence identity to SEQ ID NO:23. Further aspects relate to a polypeptide comprising an antigen binding variable region comprising a CDR3 comprising the amino acid sequence of SEQ ID NO:30 or an amino acid sequence with at least 80% sequence identity to SEQ ID NO:30. Further aspects relate to an engineered T-cell Receptor (TCR) comprising a TCR-a region and a TCR-b region, wherein the TCR-a region comprises a CDR3 with the amino acid sequence of SEQ ID NO:9 or an amino acid sequence with at least 80% sequence identity to SEQ ID NO:9 and the TCR-b region comprises a CDR3 with the amino acid sequence of SEQ ID NO:16 or an amino acid sequence with at least 80% sequence identity to SEQ ID NO:16. Further aspects relate to an engineered T-cell Receptor (TCR) comprising a TCR-a region and a TCR-b region, wherein the TCR-a region comprises a CDR3 with the amino acid sequence of SEQ ID NO:23 or an amino acid sequence with at least 80% sequence identity to SEQ ID NO:23 and the TCR-b region comprises a CDR3 with the amino acid sequence of SEQ ID NO:30 or an amino acid sequence with at least 80% sequence identity to SEQ ID NO:30.

The disclosure also relates to a fusion protein comprising a TCR of the disclosure and a CD3 binding region. In some aspects, the CD3 binding region comprises a CD3-specific fragment antigen binding (Fab), single chain variable fragment (scFv), single domain antibody, or single chain antibody.

The disclosure also relates to nucleic acids encoding the TCRs and polypeptides of the disclosure as well as compositions and cells comprising such nucleic acids, TCRs, and polypeptides. Aspects also provide for expression vectors comprising the nucleic acids of the disclosure.

Aspects of the disclosure also provide for a composition comprising at least one MHC polypeptide and a peptide of the disclosure. Further aspects of the disclosure relate to T cell receptors (TCRs) produced by methods of the disclosure and fusion proteins comprising the peptide binding regions of the TCR fused to a T cell binding region. In some aspects, the peptide binding region of the TCR comprises the alpha and beta variable regions of the TCR. In some aspects, the peptide binding region of the TCR comprises the gamma and delta variable regions of the TCR. In some aspects, the T cell binding region comprises a CD3 binding region. In some aspects, the CD3 binding region comprises an anti-CD3 Fab. In some aspects, the peptide binding region and/or T cell binding region may be an antigen binding fragment such as a scFv, Fab, single chain antibody or TCR, TCR mimic antibody, nanobody, camelid, aptamer, and/or DARPIN.

Yet further aspects relate to a peptide-specific binding molecule that bind to a peptide of the disclosure or that bind to a peptide-MHC complex. Exemplary binding molecules include antibodies, TCR mimic antibodies, scFvs, nanobodies, camellids, aptamers, and DARPINs. Related methods provide for a method comprising contacting a composition comprising at least one MHC polypeptide and a peptide of the disclosure with a composition comprising T cells and detecting T cells with bound peptide and/or MHC polypeptide by detecting a detection tag. Further aspects relate to kits comprising a peptide, nucleic acid, expression vector, or composition of the disclosure.

In some aspects, the peptide comprises at least 6 contiguous amino acids of a peptide of SEQ ID NO:1 or 2. In some aspects, the peptide comprises at least 4, 5, 6, 7, 8, or 9 contiguous amino acids of a peptide of SEQ ID NO:1 or 2. In some aspects, the peptide comprises or consists of SEQ ID NO:1 or SEQ ID NO:2. In some aspects, the peptide is 13 amino acids in length or shorter. In some aspects, the peptide has at least, at most, exactly, or consists of 7, 8, 9, 10, 11, 12, 13, 14, or 15 amino acids (or any range derivable therein). In a specific aspect, the peptide consists of 9 amino acids. In some aspects, the peptide is immunogenic. The term immunogenic may refer to the production of an immune response, such as a protective immune response. In some aspects, the peptide is modified. In some aspects, the modification comprises conjugation to a molecule. The molecule may be an antibody, a lipid, an adjuvant, or a detection moiety (tag). In some aspects, the peptide comprises 100% sequence identity to a peptide of SEQ ID NO: 1 or 2. Peptides of the disclosure also include those that have at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to a peptide of SEQ ID NO: 1 or 2. The peptides of the disclosure may have 1, 2, or 3 substitutions relative to a peptide of SEQ ID NO: 1 or 2. In some aspects, the peptide has at least or has at most 1, 2, 3, 4, or 5 substitutions relative to a peptide of SEQ ID NO: 1 or 2.

The polypeptide or TCR of the disclosure may comprise or may comprise a variable region comprising a CDR1, CDR2, and/or CDR3. In some aspects, the polypeptide comprises a T cell receptor alpha (TCR-a) variable region. In some aspects, the polypeptide comprises a TCR-a variable and constant region. In some aspects, the polypeptide further comprises a signal peptide. In some aspects, the polypeptide comprises a TCR-a variable region and a TCR-b variable region on the same polypeptide

In some aspects, the disclosure relates to a polypeptide comprising an antigen binding variable region comprising a CDR3 comprising the amino acid sequence of SEQ ID NO:9 or an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:9. In some aspects, the polypeptide comprises or the variable region comprises a CDR1 and/or CDR2 with the amino acid sequence of SEQ ID NO:7 and SEQ ID NO:8, respectively, or with at least 80% sequence identity to SEQ ID NO:7 and SEQ ID NO:8, respectively. In some aspects, the polypeptide comprises or the variable region comprises a CDR1 and/or CDR2 with the amino acid with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:7 and SEQ ID NO:8, respectively. In some aspects, the variable region comprises the amino acid sequence of SEQ ID NO:5 or an amino acid sequence with at least 70% sequence identity to SEQ ID NO:5. In some aspects, the polypeptide comprises or the variable region comprises an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:5. In some aspects, the signal peptide comprises the amino acid sequence of SEQ ID NO:6 or an amino acid sequence with at least 80% identity to SEQ ID NO:6. In some aspects, the polypeptide comprises or the signal peptide comprises an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:6.

The disclosure also provides a polypeptide comprising an antigen binding variable region comprising a CDR3 comprising the amino acid sequence of SEQ ID NO: 16 or an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:16. In some aspects, the polypeptide comprises or the variable region comprises a CDR1 and/or CDR2 with the amino acid sequence of SEQ ID NO:14 and SEQ ID NO:15, respectively, or with at least 80% sequence identity to SEQ ID NO:14 and SEQ ID NO:15, respectively. In some aspects, the polypeptide comprises or the variable region comprises a CDR1 and/or CDR2 with the amino acid with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:14 and SEQ ID NO:15, respectively. In some aspects, the variable region comprises the amino acid sequence of SEQ ID NO: 12 or an amino acid sequence with at least 70% sequence identity to SEQ ID NO: 12. In some aspects, the polypeptide comprises or the variable region comprises an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:12. In some aspects, the signal peptide comprises the amino acid sequence of SEQ ID NO:13 or an amino acid sequence with at least 80% identity to SEQ ID NO:13. In some aspects, the polypeptide comprises or the signal peptide comprises an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:13.

Aspects of the disclosure relate to a polypeptide comprising an antigen binding variable region comprising a CDR3 comprising the amino acid sequence of SEQ ID NO:23 or an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:23. In some aspects, the polypeptide comprises or the variable region comprises a CDR1 and/or CDR2 with the amino acid sequence of SEQ ID NO:21 and SEQ ID NO:22, respectively, or with at least 80% sequence identity to SEQ ID NO:21 and SEQ ID NO:22, respectively. In some aspects, the polypeptide comprises or the variable region comprises a CDR1 and/or CDR2 with the amino acid with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:21 and SEQ ID NO:22, respectively. In some aspects, the variable region comprises the amino acid sequence of SEQ ID NO: 19 or an amino acid sequence with at least 70% sequence identity to SEQ ID NO:19. In some aspects, the polypeptide comprises or the variable region comprises an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:19. In some aspects, the signal peptide comprises the amino acid sequence of SEQ ID NO:20 or an amino acid sequence with at least 80% identity to SEQ ID NO:20. In some aspects, the polypeptide comprises or the signal peptide comprises an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:20.

Aspects of the disclosure relate to a polypeptide comprising an antigen binding variable region comprising a CDR3 comprising the amino acid sequence of SEQ ID NO:30 or an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:30. In some aspects, the polypeptide comprises or the variable region comprises a CDR1 and/or CDR2 with the amino acid sequence of SEQ ID NO:28 and SEQ ID NO:29, respectively, or with at least 80% sequence identity to SEQ ID NO:28 and SEQ ID NO:29, respectively. In some aspects, the polypeptide comprises or the variable region comprises a CDR1 and/or CDR2 with the amino acid with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:28 and SEQ ID NO:29, respectively. In some aspects, the variable region comprises the amino acid sequence of SEQ ID NO:26 or an amino acid sequence with at least 70% sequence identity to SEQ ID NO:26. In some aspects, the polypeptide comprises or the variable region comprises an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:26. In some aspects, the signal peptide comprises the amino acid sequence of SEQ ID NO:27 or an amino acid sequence with at least 80% identity to SEQ ID NO:27. In some aspects, the polypeptide comprises or the signal peptide comprises an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:27.

The disclosure also describes an engineered T-cell Receptor (TCR) comprising a TCR-a region and a TCR-b region, wherein the TCR-a region comprises a CDR3 with the amino acid sequence of SEQ ID NO:9 or an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:9 and the TCR-b region comprises a CDR3 with the amino acid sequence of SEQ ID NO:16 or an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:16. In some aspects, the TCR-a region comprises a CDR1 having the amino acid sequence of SEQ ID NO:7 or having an amino acid sequence with at least 80% sequence identity to SEQ ID NO:7 and/or the TCR-b region comprises a CDR1 having the amino acid sequence of SEQ ID NO: 14 or having an amino acid sequence with at least 80% sequence identity to SEQ ID NO:14. In some aspects, the TCR comprises a CDR1 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:7. In some aspects, the TCR comprises a CDR1 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 14. In some aspects, the TCR-a region comprises a CDR2 having the amino acid sequence of SEQ ID NO:8 or having an amino acid sequence with at least 80% sequence identity to SEQ ID NO:8 and/or the TCR-b region comprises a CDR2 having the amino acid sequence of SEQ ID NO:15 or having an amino acid sequence with at least 80% sequence identity to SEQ ID NO:15. In some aspects, the TCR comprises a CDR2 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:8. In some aspects, the TCR comprises a CDR2 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:15. In some aspects, the CDR1, CDR2, and CDR3 of the TCR-a region comprise the amino acid sequence of SEQ ID NO:7, 8, and 9, respectively and wherein the CDR1, CDR3, and CDR3 of the TCR-b region comprise the amino acid sequence of SEQ ID NO:14, 15, and 16, respectively. In some aspects, the TCR-a region comprises the amino acid sequence of SEQ ID NO:5 or an amino acid sequence with at least 70% sequence identity to SEQ ID NO:5 and the TCR-b region comprises the amino acid sequence of SEQ ID NO:12 or an amino acid sequence with at least 70% sequence identity to SEQ ID NO: 12. In some aspects, the TCR comprises an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:5. In some aspects, the TCR comprises an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 12.

The disclosure also describes an engineered T-cell Receptor (TCR) comprising a TCR-a region and a TCR-b region, wherein the TCR-a region comprises a CDR3 with the amino acid sequence of SEQ ID NO:23 or an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:23 and the TCR-b region comprises a CDR3 with the amino acid sequence of SEQ ID NO:30 or an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:30. In some aspects, the TCR-a region comprises a CDR1 having the amino acid sequence of SEQ ID NO:21 or having an amino acid sequence with at least 80% sequence identity to SEQ ID NO:21 and/or the TCR-b region comprises a CDR1 having the amino acid sequence of SEQ ID NO:28 or having an amino acid sequence with at least 80% sequence identity to SEQ ID NO:28. In some aspects, the TCR comprises a CDR1 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:21. In some aspects, the TCR comprises a CDR1 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:28. In some aspects, the TCR-a region comprises a CDR2 having the amino acid sequence of SEQ ID NO:22 or having an amino acid sequence with at least 80% sequence identity to SEQ ID NO:22 and/or the TCR-b region comprises a CDR2 having the amino acid sequence of SEQ ID NO:29 or having an amino acid sequence with at least 80% sequence identity to SEQ ID NO:29. In some aspects, the TCR comprises a CDR2 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:22. In some aspects, the TCR comprises a CDR2 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:29. In some aspects, the CDR1, CDR2, and CDR3 of the TCR-a region comprise the amino acid sequence of SEQ ID NO:21, 22, and 23, respectively and wherein the CDR1, CDR3, and CDR3 of the TCR-b region comprise the amino acid sequence of SEQ ID NO:28, 29, and 30, respectively. In some aspects, the TCR-a region comprises the amino acid sequence of SEQ ID NO:19 or an amino acid sequence with at least 70% sequence identity to SEQ ID NO:19 and the TCR-b region comprises the amino acid sequence of SEQ ID NO:26 or an amino acid sequence with at least 70% sequence identity to SEQ ID NO:26. In some aspects, the TCR comprises an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:19. In some aspects, the TCR comprises an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:26.

Aspects of the disclosure relate to a nucleic acid encoding a TCR-a region and/or a TCR-b region, wherein the TCR-a region comprises a CDR3 with the amino acid sequence of SEQ ID NO:9 or an amino acid sequence with at least 80% sequence identity to SEQ ID NO:9 and the TCR-b region comprises a CDR3 with the amino acid sequence of SEQ ID NO:16 or an amino acid sequence with at least 80% sequence identity to SEQ ID NO:16. In some aspects, the nucleic acid encodes for a TCR-a region comprising a CDR3 with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:9 and/or a TCR-b region comprising a CDR3 with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:16. In some aspects, the nucleic acid encodes for a TCR-a region comprising a CDR1 having the amino acid sequence of SEQ ID NO:7 or having an amino acid sequence with at least 80% sequence identity to SEQ ID NO:7 and/or a TCR-b region comprising a CDR1 having the amino acid sequence of SEQ ID NO:14 or having an amino acid sequence with at least 80% sequence identity to SEQ ID NO:14. In some aspects, the nucleic acid encodes for a TCR-a region comprising a CDR1 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:7 and/or a TCR-b region comprising a CDR1 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:14. In some aspects, the nucleic acid encodes for TCR-a region comprising a CDR2 having the amino acid sequence of SEQ ID NO:8 or having an amino acid sequence with at least 80% sequence identity to SEQ ID NO:8 and/or a TCR-b region comprising a CDR2 having the amino acid sequence of SEQ ID NO: 15 or having an amino acid sequence with at least 80% sequence identity to SEQ ID NO:15. In some aspects, the nucleic acid encodes for TCR-a region comprising a CDR2 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:8 and/or a TCR-b region comprising a CDR2 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 15. In some aspects, the nucleic acid encodes a TCR-a variable region having the amino acid sequence of SEQ ID NO:5 or an amino acid sequence with at least 70% sequence identity to SEQ ID NO:5 and/or a TCR-b variable region having the amino acid sequence of SEQ ID NO: 12 or an amino acid sequence with at least 70% sequence identity to SEQ ID NO:12. In some aspects, the nucleic acid encodes a TCR-a variable region having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:5 and/or a TCR-b variable region having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 12. In some aspects, the nucleic acid encodes a TCR-a region having the amino acid sequence of SEQ ID NO:4 or an amino acid sequence with at least 70% sequence identity to SEQ ID NO:4 and/or a TCR-b region chain having the amino acid sequence of SEQ ID NO:11 or an amino acid sequence with at least 70% sequence identity to SEQ ID NO:11. In some aspects, the nucleic acid encodes a TCR-a region comprising an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:4 and/or a TCR-b region having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:11. In some aspects, the nucleic acid comprises SEQ ID NO:3, 10, and/or a fragment thereof. In some aspects, the nucleic acid has at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:3, 10, and/or a fragment thereof.

Aspects of the disclosure relate to a nucleic acid encoding a TCR-a region and/or a TCR-b region, wherein the TCR-a region comprises a CDR3 with the amino acid sequence of SEQ ID NO:23 or an amino acid sequence with at least 80% sequence identity to SEQ ID NO:23 and the TCR-b region comprises a CDR3 with the amino acid sequence of SEQ ID NO:30 or an amino acid sequence with at least 80% sequence identity to SEQ ID NO:30. In some aspects, the nucleic acid encodes for a TCR-a region comprising a CDR3 with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:23 and/or a TCR-b region comprising a CDR3 with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:30. In some aspects, the nucleic acid encodes for a TCR-a region comprising a CDR1 having the amino acid sequence of SEQ ID NO:21 or having an amino acid sequence with at least 80% sequence identity to SEQ ID NO:21 and/or a TCR-b region comprising a CDR1 having the amino acid sequence of SEQ ID NO:28 or having an amino acid sequence with at least 80% sequence identity to SEQ ID NO:28. In some aspects, the nucleic acid encodes for a TCR-a region comprising a CDR1 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:21 and/or a TCR-b region comprising a CDR1 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:28. In some aspects, the nucleic acid encodes for TCR-a region comprising a CDR2 having the amino acid sequence of SEQ ID NO:22 or having an amino acid sequence with at least 80% sequence identity to SEQ ID NO:22 and/or a TCR-b region comprising a CDR2 having the amino acid sequence of SEQ ID NO:29 or having an amino acid sequence with at least 80% sequence identity to SEQ ID NO:29. In some aspects, the nucleic acid encodes for TCR-a region comprising a CDR2 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:22 and/or a TCR-b region comprising a CDR2 having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:29. In some aspects, the nucleic acid encodes a TCR-a variable region having the amino acid sequence of SEQ ID NO:19 or an amino acid sequence with at least 70% sequence identity to SEQ ID NO: 19 and/or a TCR-b variable region having the amino acid sequence of SEQ ID NO:26 or an amino acid sequence with at least 70% sequence identity to SEQ ID NO:26. In some aspects, the nucleic acid encodes a TCR-a variable region having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:19 and/or a TCR-b variable region having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:26. In some aspects, the nucleic acid encodes a TCR-a region having the amino acid sequence of SEQ ID NO:18 or an amino acid sequence with at least 70% sequence identity to SEQ ID NO:18 and/or a TCR-b region chain having the amino acid sequence of SEQ ID NO:25 or an amino acid sequence with at least 70% sequence identity to SEQ ID NO:25. In some aspects, the nucleic acid encodes a TCR-a region comprising an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 18 and/or a TCR-b region having an amino acid sequence with at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:25. In some aspects, the nucleic acid comprises SEQ ID NO: 17, 24, and/or a fragment thereof. In some aspects, the nucleic acid has at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 17, 24, and/or a fragment thereof.

Nucleic acids of the disclosure may encode for a TCR-a region comprising a CDR1. CDR2, and CDR3 and/or a TCR-b region comprising a CDR1, CDR2, and CDR3. In some aspects, the nucleic acid comprises at least one TCR-a (TRA) and at least one TCR-b (TRB) gene on the same nucleic acid. The nucleic acid may also encode a bi-specific polypeptide comprising a TCR of the disclosure and a second antigen-binding region. The second antigen binding region may be a scFv, single chain antibody, single domain antibody, or diabody, for example. In some aspects, the nucleic acid is polycistronic. In some aspects, the nucleic acid comprises an internal ribosome entry site (IRES) or a 2A cleavable linker. In some aspects, the nucleic acid comprises a cDNA encoding the TCR-a and/or TCR-b genes. In some aspects, the nucleic acid further encodes for a polypeptide comprising a CD3 binding region. In some aspects, the CD3 binding region comprises a CD3-specific fragment antigen binding (Fab), single chain variable fragment (scFv), single domain antibody, or single chain antibody. In some aspects, the vector comprises a promoter that directs the expression of the nucleic acid. In some aspects, the promoter comprises a murine stem cell virus (MSCV) promoter. In some aspects, the vector comprises the TCR-a and TCR-b genes. The nucleic acids of the disclosure may also be further defined as cDNAs (complementary DNA).

Also disclosed a a method of making an engineered cell comprising transferring a nucleic acid or vector of the disclosure into a cell. In some aspects, the method further comprises culturing the cell in media, incubating the cell at conditions that allow for the division of the cell, screening the cell, and/or freezing the cell.

In some aspects, the cell of the disclosure comprises a stem cell, a progenitor cell, an immune cell, or a natural killer (NK) cell. In some aspects, the cell comprises a hematopoietic stem or progenitor cell, a T cell, a cell differentiated from mesenchymal stem cells (MSCs) or an induced pluripotent stem cell (iPSC). In some aspects, the cell is isolated or derived from peripheral blood mononuclear cell (PBMCs). In some aspects, the T cell comprises a cytotoxic T lymphocyte (CTL), a CD8+ T cell, a CD4+ T cell, an invariant NK T (INKT) cell, a gamma-delta T cell, a NKT cell, a regulatory T cell, or a CD4+T regulatory cell. In some aspects, the cell is isolated from a cancer patient. In some aspects, the composition has been determined to be serum-free, mycoplasma-free, endotoxin-free, and/or sterile.

In some aspects, the pharmaceutical composition is formulated for parenteral administration, intravenous injection, intramuscular injection, inhalation, or subcutaneous injection. In some aspects, the peptide is comprised in a liposome, lipid-containing nanoparticle, or in a lipid-based carrier. In some aspects, the pharmaceutical preparation is formulated for injection or inhalation as a nasal spray. In some aspects, the compositions of the disclosure are formulated as a vaccine. In some aspects, the composition further comprises an adjuvant.

In some aspects regarding the dendritic cells of the disclosure, the dendritic cell comprises a mature dendritic cell. In some aspects, the cell is a cell with an HLA-A type. The HLA may be a HLA-A, HLA-B, or HLA-C. In some aspects, the cell is an HLA-A24 type. In some aspects, the cell is an HLA-A11 type. In some aspects, the cell is an HLA-A01, HLA-A02, HLA-A11, HLA-A24, HLA-B07, HLA-B08, HLA-B15, or HLA-B40.

In some aspects, the method further comprises isolating the expressed peptide or polypeptide. In some aspects, the T cell comprises a CD8+ T cell. In some aspects, the T cell is a CD4+ T cell, a Th1, Th2, Th17, Th9, or Tfh T cell, a cytotoxic T cell, a memory T cell, a central memory T cell, or an effector memory T cell.

In method aspects of the disclosure, contacting is further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface. In some aspects, the APCs are dendritic cells.

In aspects of the disclosure, the immune effector cells are T cells, peripheral blood lymphocytes, natural killer (NK) cells, invariant NK cells, or NKT cells. The immune effector cells may be ones that have been differentiated from mesenchymal stem cell (MSC) or induced pluripotent stem (iPS) cells. The T cell aspects include T cells that are further defined as CD8+ T cells, CD4+ T cells, or γδ T cells. In certain aspects, the T cells are cytotoxic T lymphocytes (CTLs).

In some aspects, the subject described in the methods of the disclosure is a human. In some aspects, the subject is a laboratory animal. In some aspects, the subject is a mouse, rat, pig, horse, rabbit, or guinea pig. Aspects also relate to methods comprising further administration of at least a second therapeutic agent. In some aspects, the second therapeutic agent is an anti-cancer agent. Treating, as defined in the methods of the disclosure, may comprise one or more of reducing tumor size; increasing the overall survival rate; reducing the risk of recurrence of the cancer; reducing the risk of progression; and/or increasing the chance of progression-free survival, relapse-free survival, and/or recurrence-free survival.

The biological sample in methods of the disclosure may comprise a blood sample or a fraction thereof. In some aspects, the biological sample comprises lymphocytes. In some aspects, the biological sample comprises a fractionated sample comprising lymphocytes. In some aspects, the biological sample is one described herein.

The TCR of the disclosure may be further defined as a single-chain TCR. In some aspects, the TCR-a and TCR-b regions are on the same polypeptide. In some aspects, the polypeptide is further defined as recombinant. In some aspects, the TCR-a and TCR-b regions are on different polypeptides. Also provided are polypeptides comprising a TCR of the disclosure. In some aspects, the polypeptides may be bi-specific. For example, the polypeptide may have one peptide specificity from the TCR and a second specificity from another antigen binding region. The further antigen binding region may be another TCR, a scFv, a diabody, or a single chain antibody, for example.

In some aspects, the composition of the disclosure comprises a MHC polypeptide and a peptide of the disclosure and wherein the MHC polypeptide and/or peptide is conjugated to a detection tag. As such, suitable detection tags include, but are not limited to radioisotopes, fluorochromes, chemiluminescent compounds, dyes, and proteins, including enzymes. The tag may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent tags that produce signals include, but are not limited to bioluminescence and chemiluminescence. Examples of suitable fluorescent tags include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.). Detection tags also include streptavidin or it's binding partner, biotin.

In some aspects, the MHC polypeptide and peptide are operatively linked. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. Accordingly, aspects of the disclosure relate to wherein the MHC polypeptide and peptide are operatively linked through a peptide bond. Further aspects relate to wherein the MHC polypeptide and peptide are operatively linked through van der Waals forces. The peptide-MHC may be operatively linked to form a pMHC complex. In some aspects, at least two pMHC complexes are operatively linked together. Other aspects include, include at least, or include at most 2, 3, 4, 5, 6, 7, 8, 9, or 10 pMHC complexes operatively linked to each other. In some aspects, at least two MHC polypeptides are linked to one peptide. In other aspects, the average ratio of MHC polypeptides to peptides is 1:1 to 4:1. In some aspects, the ratio or average ratio is at least, at most, or about 1, 2, 3, 4, 5, or 6 to about 1, 2, 3, 4, 5, or 6 (or any derivable range therein).

In some of the aspects of the disclosure, the peptide is complexed with MHC In some aspects, the MHC comprises HLA-A type. The MHC may be further defined as HLA-A3 or HLA-A11 type. The peptides may be loaded onto dendritic cells, lymphoblastoid cells, peripheral blood mononuclear cells (PBMCs), artificial antigen presentation cells (aAPC) or artificial antigen presenting surfaces. In some aspects, the artificial antigen presenting surface comprises a MHC polypeptide conjugated or linked to a surface. Exemplary surfaces include a bead, microplate, glass slide, or cell culture plate.

Method of the disclosure may further comprise counting the number of T cells bound with peptide and/or MHC. The composition comprising T cells may be isolated from a patient having or suspected of having cancer. The cancer may comprise a peptide-specific cancer, wherein the peptide of SEQ ID NO: 1 or 2 or a peptide of the disclosure. The subject may be a subject that has been diagnosed and/or determined to have a cancer. The subject or patient may also be one that has been characterized as having a peptide-specific cancer, such as a peptide of the disclosure or a peptide of SEQ ID NO:1 or 2. In some aspects, the method further comprises sorting the number of T cells bound with peptide and/or MHC. Methods of the disclosure may also comprise or further comprise sequencing one or more TCR genes from T cells bound with peptide and/or MHC. In some aspects, the method comprises or further comprises sequencing the TCR alpha and/or beta gene(s) from a TCR, such as a TCR that binds to a peptide of the disclosure. Methods may also comprise or further comprise grouping of lymphocyte interactions by paratope hotspots (GLIPH) analysis. This is further described in Glanville et al., Nature. 2017 Jul. 6; 547(7661): 94-98, which is herein incorporated by reference.

The compositions of the disclosure may be serum-free, mycoplasma-free, endotoxin-free, and/or sterile. The methods may further comprise culturing cells of the disclosure in media, incubating the cells at conditions that allow for the division of the cell, screening the cells, and/or freezing the cells. In some aspects, the method further comprises isolating the expressed peptide or polypeptide from a cell of the disclosure.

Methods of the disclosure may comprise or further comprise screening the dendritic cell for one or more cellular properties. In some aspects, the method further comprises contacting the cell with one or more cytokines or growth factors. The one or more cytokines or growth factors may comprise GM-CSF. In some aspects, the cellular property comprises cell surface expression of one or more of CD86, HLA, and CD14. In some aspects, the dendritic cell is derived from a CD34+ hematopoietic stem or progenitor cell.

The contacting in the methods of the disclosure may be further defined as co-culturing the starting population of immune effector cells with antigen presenting cells (APCs), wherein the APCs present the peptide on their surface. In a particular aspect, the APCs are dendritic cells. In some aspects, the dendritic cell is derived from a peripheral blood monocyte (PBMC). In some aspects, the dendritic cells are isolated from PBMCs. In some aspects, the dendritic cells are cells in which the DCs are derived from are isolated by leukaphereses.

Peptide-MHC (pMHC) complexes in the aspects of the disclosure may be made by contacting a peptide of the disclosure with a MHC complex. In some aspects, the peptide is expressed in the cell and binds to endogenous MHC complex to form a pMHC. In some aspects, peptide exchange is used to make the pMHC complex. For example, cleavable peptides, such as photocleavable peptides may be designed that bind to and stabilize the MHC. Cleavage of the peptide (eg, by irradiation for photocleavable peptides) dissociates the peptide from the HLA complex and results in an empty HLA complex that disintegrates rapidly, unless UV exposure is performed in the presence of a “rescue peptide.” Thus, the peptides of the disclosure may be used as “rescue peptides” in the peptide exchange procedure. Further aspects of the disclosure relate to pMHC complexes comprising a peptide of the disclosure. The pMHC complex may be operatively linked to a solid support or may be attached to a detectable moiety, such as a fluorescent molecule, a radioisotope, or an antibody. Further aspects of the disclosure relate to peptide-MHC multimeric complexes that include, include at least or include at most 1, 2, 3, 4, 5, or 6 peptide-MHC molecules operatively linked together. The linkage may be covalent, such as through a peptide bond, or non-covalent. In some aspects, pMHC molecules may be bound to a biotin molecule. Such pMHC molecules may be multimerized through binding to a streptavidin molecule. pMHC multimers may be used to detect antigen-specific T cells or TCR molecules that are in a composition or in a tissue. In some aspects, the multimers may be used to detect peptide-specific T cells in situ or in a biopsy sample. In further aspects, multimers may be bound to a solid support or deposited on a solid support, such as an array or slide. Cells may then be added to the slide, and detection of the binding between the pMHC multimer and cell may be conducted. Accordingly, the pMHC molecules and multimers of the disclosure may be used to detect and diagnose cancer in subjects or to determine immune responses in individuals with cancer.

In some aspects of the disclosure, obtaining, as defined in the methods described herein, comprises isolating the starting population of immune effector cells from peripheral blood mononuclear cells (PBMCs). In some aspects, the starting population of immune effector cells is obtained from a subject. The subject may be one that has a cancer, such as a peptide-specific cancer. In some aspects, the subject has been determined to have a cancer that expresses a peptide of the disclosure. In some aspects, the cancer comprises leukemia, lung cancer, or skin cancer. The cancer may also be further defined as a solid tumor. In a specific aspect, the cancer comprises lung cancer. The lung cancer may comprise adenocarcinoma or squamous cell carcinoma. In some aspects, the methods of the disclosure comprise or further comprise introducing the peptides or a nucleic acid encoding the peptide into the dendritic cells prior to the co-culturing. The introduction of the peptide may be done by transfecting or infecting dendritic cells with a nucleic acid encoding the peptide or by incubating the peptide with the dendritic cells. In some aspects, the peptide or nucleic acids encoding the peptide are introduced by electroporation. Other methods of transfer of nucleic acids are known in the art, such as lipofection, calcium phosphate transfection, transfection with DEAE-dextran, microinjection, and virus-mediated transduction. In some aspects, the peptide or nucleic acids encoding the peptide are introduced by adding the peptide or nucleic acid encoding the peptide to the dendritic cell culture media. In some aspects, the immune effector cells are co-cultured with a second population of dendritic cells into which the peptide or the nucleic acid encoding the peptide has been introduced. In some aspects, a population of CD4-positive or CD8-positive and peptide MHC tetramer-positive T cells are purified from the immune effector cells following the co-culturing. In some aspects, the population of CD4-positive or CD8-positive and peptide MHC tetramer-positive T cells are purified by fluorescence activated cell sorting (FACS). In some aspects, a clonal population of peptide-specific immune effector cells are generated by limiting or serial dilution followed by expansion of individual clones by a rapid expansion protocol.

In some aspects, purifying further comprises generation of a clonal population of peptide-specific immune effector cells by limiting or serial dilution of sorted cells followed by expansion of individual clones by a rapid expansion protocol. In some aspects, methods of the disclosure comprise or further comprise cloning of a T cell receptor (TCR) from the clonal population of peptide-specific immune effector cells. In some aspects, the term isolating in the methods of the disclosure is defined as cloning of a T cell receptor (TCR) from the clonal population of peptide-specific immune effector cells. In some aspects, cloning of the TCR is cloning of a TCR alpha and a beta chain. In some aspects, the TCR is cloned using a 5′-Rapid amplification of cDNA ends (RACE) method. In some aspects, the TCR alpha and beta chains are cloned using a 5′-Rapid amplification of cDNA ends (RACE) method. In some aspects, the cloned TCR is subcloned into an expression vector. In some aspects, the expression vector comprises a linker domain between the TCR alpha sequence and TCR beta sequence. In some aspects, the expression vector is a retroviral or lentiviral vector. The vector may also be an expression vector described herein. The linker domain may comprise a sequence encoding one or more peptide cleavage sites. The one or more cleavage sites may be a Furin cleavage site and/or a P2A cleavage site. In some aspects, the TCR alpha sequence and TCR beta sequence are linked by an IRES sequence.

A host cell of the disclosure may be transduced with an expression vector to generate an engineered cell that expresses the TCR alpha and/or beta chains. In some aspects, the host cell is an immune cell. The immune cell may be a T cell and the engineered cell may be referred to as an engineered T cell. The T cell may be type of T cell described herein, such as a CD8+ T cell, CD4+ T cell, or γδ T cell. In some aspects, the starting population of immune effector cells is obtained from a subject having a cancer or a peptide-specific cancer and the host cell is allogeneic or autologous to the subject. In some aspects, the peptide-specific T cells are autologous or allogeneic. In some aspects, a population of CD4-positive or CD8-positive and peptide MHC tetramer-positive engineered T cells are purified from the transduced host cells. In some aspects, a clonal population of peptide-specific engineered T cells are generated by limiting or serial dilution followed by expansion of individual clones by a rapid expansion protocol. In some aspects, purifying in the methods of the disclosure is defined as purifying a population of CD4-positive or CD8-positive and peptide MHC tetramer-positive T cells from the immune effector cells following the co-culturing.

In some aspects, the peptide is linked to a solid support. In some aspects, the peptide is conjugated to the solid support or is bound to an antibody that is conjugated to the solid support. In some aspects, the solid support comprises a microplate, a bead, a glass surface, a slide, or a cell culture dish. In some aspects, the solid support comprises a nanofluidic chip. In some aspects, detecting T cell responses comprises detecting the binding of the peptide to the T cell or TCR. In some aspects, detecting T cell responses comprises an ELISA, ELISPOT, or a tetramer assay.

Kit aspects of the disclosure may comprise a peptide of the disclosure in a container. The peptide may be comprised in a pharmaceutical preparation. In some aspects, the pharmaceutical preparation is formulated for parenteral administration or inhalation. In some aspects, the peptide is comprised in a cell culture media.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.”

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z.” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments and aspects are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments and aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Hormad1 HLA-A1101 restricted peptide identification using MHC immunoaffinity precipitation and mass spectrometric analysis.

FIG. 2. HORMAD1 HLA-A11 restricted peptide (HMD-375, SSSQESVPK—SEQ ID NO:2) CTL generation.

FIG. 3. HMD-375 peptide specific CTL cell lines epitope recognition affinity detection with peptide titration assay.

FIG. 4A-D. Functional validation of HMD-375 peptide specific CTL cell lines. Cell targets include: (A) K562-A11 cell lines forced expressing eGFP or HORMAD1; (B) H1299 (HLA-A2−, HORMAD1+) and H1299-A11 (HLA-A11 forced expressing) (C) H1623 (HLA-A11+, HORMAD1+) and H647 (HLA-A11+, HORMAD1−) (D) M14-eGFP (HLA-A11+, HORMAD1−, eGFP forced expressing) and M14-HMD (HLA-A11+, HORMAD1 forced expressing).

FIG. 5. HMD-375 specific TCR-T cell line generation.

FIG. 6. The recognition affinity detection of HMD-375 specific TCR-T cell lines with peptide titration assay.

FIG. 7A-D. Functional validation of HMD-375 specific TCR-T cell lines. Cell targets include: (A) K562-A11 cell lines forced expressing eGFP or HORMAD1; (B) H1299 (HLA-A2−, HORMAD1+) and H1299-A11 (HLA-A11 forced expressing); (C) H1623 (HLA-A11+, HORMAD1+) and H647 (HLA-A11+, HORMAD1−); (D) M14-eGFP (HLA-A11+, HORMAD1−, eGFP forced expressing) and M14-HMD (HLA-A11+, HORMAD1 forced expressing).

FIG. 8A-B. Functional validation of HMD-375 specific TCR-T cell lines using intracellular cytokine staining (ICS) assay showing IFN-γ (A) and TNF-α (B) cytokine staining.

FIG. 9. KIF2C expression level in human normal tissues (GTEx database).

FIG. 10. KIF2C expression level in human cancer tissues (TCGA database).

FIG. 11. HLA-A2402 restricted peptide—IYNGKLFDL (SEQ ID NO:1), was eluted from K562-A24 cells and identified with mass spectrometry. The sequence on the graph corresponds to SEQ ID NO:1.

FIG. 12. KIF2C-404 peptide (IYNGKLFDL—SEQ ID NO:2) specific CTL generation.

FIG. 13. KIF2C-404 peptide specific CTL cell lines epitope recognition affinity detection with peptide titration assay.

FIG. 14A-E. Functional validation of KIF2C-404 peptide specific CTL cell lines. Cell targets include (A) H650; (B) U118G; (C) LN18; (D) H1299; (E) Panc 03.27.

FIG. 15. KIF2C-404 specific TCR-T cell line generation.

FIG. 16. Functional validation of KIF2C-404 peptide specific TCR T cell lines.

### DETAILED DESCRIPTION OF THE INVENTION

### I. Immunotherapies Using Peptides of the Disclosure

A peptide as described herein (e.g., a peptide of SEQ ID NO: 1 or 2) may be used for immunotherapy of a cancer. For example, a peptide of SEQ ID NO:1 or 2 may be contacted with or used to stimulate a population of T cells to induce proliferation of the T cells that recognize or bind said peptide. In other aspects, a peptide of the disclosure may be administered to a subject, such as a human patient, to enhance the immune response of the subject against a cancer.

A peptide of the disclosure may be included in an active immunotherapy (e.g., a cancer vaccine) or a passive immunotherapy (e.g., an adoptive immunotherapy). Active immunotherapies include immunizing a subject with a purified peptide antigen or an immunodominant peptide (native or modified); alternatively, antigen presenting cells pulsed with a peptide of the disclosure (or transfected with genes encoding an antigen comprising the peptide) may be administered to a subject. The peptide may be modified or contain one or more mutations such as, e.g., a substitution mutation. Passive immunotherapies include adoptive immunotherapies. Adoptive immunotherapies generally involve administering cells to a subject, wherein the cells (e.g., cytotoxic T cells) have been sensitized in vitro to a peptide of the disclosure (sec, e.g., U.S. Pat. No. 7,910,109).

In some aspects, flow cytometry may be used in the adoptive immunotherapy for rapid isolation of human tumor antigen-specific T-cell clones by using, e.g., T-cell receptor (TCR) VB antibodies in combination with carboxyfluorescein succinimidyl ester (CFSE)-based proliferation assay. Sec, e.g., Lee et al., J. Immunol. Methods, 331:13-26, 2008, which is incorporated by reference for all purposes. In some aspects, tetramer-guided cell sorting may be used such as, e.g., the methods described in Pollack, et al., J Immunother Cancer. 2014; 2: 36, which is herein incorporated by reference for all purposes. Various culture protocols are also known for adoptive immunotherapy and may be used in aspects of the disclosure. In some aspects, cells may be cultured in conditions which do not require the use of antigen presenting cells (e.g., Hida et al., Cancer Immunol. Immunotherapy, 51:219-228, 2002, which is incorporated by reference). In other aspects, T cells may be expanded under culture conditions that utilize antigen presenting cells, such as dendritic cells (Nestle et al., 1998, incorporated by reference), and in some aspects artificial antigen presenting cells may be used for this purpose (Maus et al., 2002 incorporated by reference). Additional methods for adoptive immunotherapy are disclosed in Dudley et al. (2003), which is incorporated by reference, that may be used with aspects of the current disclosure. Various methods are known and may be used for cloning and expanding human antigen-specific T cells (see, e.g., Riddell et al., 1990, which is herein incorporated by reference).

In certain aspects, the following protocol may be used to generate T cells that selectively recognize peptides of the disclosure. Peptide-specific T-cell lines may be generated from normal donors or HLA-restricted normal donors and patients using methods previously reported (Hida et al., 2002). Briefly, PBMCs (1×10⁵ cells/well) can be stimulated with about 10 μg/ml of each peptide in quadruplicate in a 96-well, U-bottom-microculture plate (Corning Incorporated, Lowell, MA) in about 200 μl of culture medium. The culture medium may consist of 50% AIM-V medium (Invitrogen), 50% RPMI1640 medium (Invitrogen), 10% human AB serum (Valley Biomedical, Winchester, VA), and 100 IU/ml of interleukin-2 (IL-2). Cells may be restimulated with the corresponding peptide about every 3 days. After 5 stimulations, T cells from each well may be washed and incubated with T2 cells in the presence or absence of the corresponding peptide. After about 18 hours, the production of interferon (IFN)-γ may be determined in the supernatants by ELISA. T cells that secret large amounts of IFN-γ may be further expanded by a rapid expansion protocol (Riddell et al., 1990; Yee et al., 2002b).

In some aspects, an immunotherapy may utilize a peptide of the disclosure that is associated with a cell penetrator, such as a liposome or a cell penetrating peptide (CPP). Antigen presenting cells (such as dendritic cells) pulsed with peptides may be used to enhance antitumour immunity (Celluzzi et al., 1996; Young et al., 1996). Liposomes and CPPs are described in further detail below. In some aspects, an immunotherapy may utilize a nucleic acid encoding a peptide of the disclosure, wherein the nucleic acid is delivered, e.g., in a viral vector or non-viral vector.

In some aspects, a peptide of the disclosure may be used in an immunotherapy to treat cancer in a mammalian subject, such as a human patient.

### II. Engineered T Cell Receptors

T-cell receptors comprise two different polypeptide chains, termed the T-cell receptor α (TCRα) and β (TCRβ) chains, linked by a disulfide bond. These α:β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated γ and δ. Both types of T cell receptor differ from the membrane-bound immunoglobulin that serves as the B-cell receptor: a T cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

The three-dimensional structure of the T-cell receptor has been determined. The structure is indeed similar to that of an antibody Fab fragment, as was suspected from earlier studies on the genes that encoded it. The T-cell receptor chains fold in much the same way as those of a Fab fragment, although the final structure appears a little shorter and wider. There are, however, some distinct differences between T-cell receptors and Fab fragments. The most striking difference is in the Cα domain, where the fold is unlike that of any other immunoglobulin-like domain. The half of the domain that is juxtaposed with the Cβ domain forms a B sheet similar to that found in other immunoglobulin-like domains, but the other half of the domain is formed of loosely packed strands and a short segment of a helix. The intramolecular disulfide bond, which in immunoglobulin-like domains normally joins two β strands, in a Cα domain joins a β strand to this segment of a helix.

There are also differences in the way in which the domains interact. The interface between the V and C domains of both T-cell receptor chains is more extensive than in antibodies, which may make the hinge joint between the domains less flexible. And the interaction between the Cα and CB domains is distinctive in being assisted by carbohydrate, with a sugar group from the Cα domain making a number of hydrogen bonds to the CB domain. Finally, a comparison of the variable binding sites shows that, although the complementarity-determining region (CDR) loops align fairly closely with those of antibody molecules, there is some displacement relative to those of the antibody molecule. This displacement is particularly marked in the Vα CDR2 loop, which is oriented at roughly right angles to the equivalent loop in antibody V domains, as a result of a shift in the β strand that anchors one end of the loop from one face of the domain to the other. A strand displacement also causes a change in the orientation of the Vβ CDR2 loop in two of the seven Vβ domains whose structures are known. As yet, the crystallographic structures of seven T cell receptors have been solved to this level of resolution.

Aspects of the disclosure relate to engineered T cell receptors. The term “engineered” refers to T cell receptors that have TCR variable regions grafted onto TCR constant regions to make a chimeric polypeptide that binds to peptides and antigens of the disclosure. In certain aspects, the TCR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

In some aspects, the TCR comprises non-TCR sequences. Accordingly, certain aspects relate to TCRs with sequences that are not from a TCR gene. In some aspects, the TCR is chimeric, in that it contains sequences normally found in a TCR gene, but contains sequences from at least two TCR genes that are not necessarily found together in nature.

In some aspects the engineered TCRs of the disclosure comprise an aspect as shown below:

TABLE 1
TCR Aspects

| | | SEQ ID |
|---|---|---|
| Description | Sequence | NO: |
| KIF2C-404 F11 TCR | ATGGAGACCCTCTTGGGCCTGCTTATCCTTTGGCTGC | 3 |
| (TRAV21*02F- | AGCTGCAATGGGTGAGCAGCAAACAGGAGGTGACA | |
| TRBV11-2*03F) DNA | CAGATTCCTGCAGCTCTGAGTGTCCCAGAAGGAGAA | |
| sequence: Alpha Chain | AACTTGGTTCTCAACTGCAGTTTCACTGATAGCGCT | |
| | ATTTACAACCTCCAGTGGTTTAGGCAGGACCCTGGG | |
| | AAAGGTCTCACATCTCTGTTGCTTATTCAGTCAAGTC | |
| | AGAGAGAGCAAACAAGTGGAAGACTTAATGCCTCG | |
| | CTGGATAAATCATCAGGACGTAGTACTTTATACATT | |
| | GCAGCTTCTCAGCCTGGTGACTCAGCCACCTACCTC | |
| | TGTGCTGTGGATACTGGAGCCAATAGTAAGCTGACA | |
| | TTTGGAAAAGGAATAACTCTGAGTGTTAGACCAGAT | |
| | ATCCAGAACCCTGACCCTGCCGTGTACCAGCTGAGA | |
| | GACTCTAAATCCAGTGACAAGTCTGTCTGCCTATTC | |
| | ACCGATTTTGATTCTCAAACAAATGTGTCACAAAGT | |
| | AAGGATTCTGATGTGTATATCACAGACAAAACTGTG | |
| | CTAGACATGAGGTCTATGGACTTCAAGAGCAACAGT | |
| | GCTGTGGCCTGGAGCAACAAATCTGACTTTGCATGT | |
| | GCAAACGCCTTCAACAACAGCATTATTCCAGAAGAC | |
| | ACCTTCTTCCCCAGCCCAGAAAGTTCCTGTGATGTC | |
| | AAGCTGGTCGAGAAAAGCTTTGAAACAGATACGAA | |
| | CCTAAACTTTCAAAACCTGTCAGTGATTGGGTTCCG | |
| | AATCCTCCTCCTGAAAGTGGCCGGGTTTAATCTGCT | |
| | CATGACGCTGCGGCTGTGGTCCAGCTAA | |
| KIF2C-404 F11 TCR | METLLGLLILWLQLQWVSSKQEVTQIPAALSVPEGENL | 4 |
| (TRAV21*02F- | VLNCSFTDSAIYNLQWFRQDPGKGLTSLLLIQSSQREQ | |
| TRBV11-2*03F) Protein | TSGRLNASLDKSSGRSTLYIAASQPGDSATYLCAVDTG | |
| sequence Alpha Chain | ANSKLTFGKGITLSVRPDIQNPDPAVYQLRDSKSSDKS | |
| | VCLFTDFDSQTNVSQSKDSDVYITDKTVLDMRSMDFK | |
| | SNSAVAWSNKSDFACANAFNNSIIPEDTFFPSPESSCDV | |
| | KLVEKSFETDTNLNFQNLSVIGFRILLLKVAGFNLLMT | |
| | LRLWSS | |
| KIF2C-404 F11 TCR | KQEVTQIPAALSVPEGENLVLNCSFTDSAIYNLQWFRQ | 5 |
| (TRAV21*02F- | DPGKGLTSLLLIQSSQREQTSGRLNASLDKSSGRSTLYI | |
| TRBV11-2*03F) | AASQPGDSATYLCAVDTGANSKLTFGKGITLSVRPD | |
| Variable region: Alpha | | |
| Chain | | |
| KIF2C-404 F11 TCR | METLLGLLILWLQLQWVSS | 6 |
| (TRAV21*02F- | | |
| TRBV11-2*03F) Leader | | |
| peptide: Alpha Chain | | |
| KIF2C-404 F11 TCR | DSAIYN | 7 |
| (TRAV21*02F- | | |
| TRBV11-2*03F) Alpha | | |
| Chain CDR1 | | |
| KIF2C-404 F11 TCR | IQSSQRE | 8 |
| (TRAV21*02F- | | |
| TRBV11-2*03F) Alpha | | |
| Chain CDR2 | | |
| KIF2C-404 F11 TCR | AVDTGANSKLT | 9 |
| (TRAV21*02F- | | |
| TRBV11-2*03F) Alpha | | |
| Chain CDR3 | | |
| KIF2C-404 F11 TCR | ATGGGCACCAGGCTCCTCTGCTGGGCGGCCCTCTGT | 10 |
| (TRAV21*02F- | CTCCTGGGAGCAGAACTCACAGAAGCTGGAGTTGCC | |
| TRBV11-2*03F) DNA | CAGTCTCCCAGATATAAGATTATAGAGAAAAGGCA | |
| sequence: Beta Chain | GAGTGTGGCTTTTTGGTGCAATCCTATATCTGGCCAT | |
| | GCTACCCTTTACTGGTACCAGCAGATCCTGGGACAG | |
| | GGCCCAAAGCTTCTGATTCAGTTTCAGAATAACGGT | |
| | GTAGTGGATGATTCACAGTTGCCTAAGGATCGATTT | |
| | TCTGCAGAGAGGCTCAAAGGAGTAGACTCCACTCTC | |
| | AAGATCCAACCTGCAAAGCTTGAGGACTCGGCCGTG | |
| | TATCTCTGTGCCAGCAGCTATCATAGCTATGGCTAC | |
| | ACCTTCGGTTCGGGGACCAGGTTAACCGTTGTAGAG | |
| | GACCTGAACAAGGTGTTCCCACCCGAGGTCGCTGTG | |
| | TTTGAGCCATCAGAAGCAGAGATCTCCCACACCCAA | |
| | AAGGCCACACTGGTGTGCCTGGCCACAGGCTTCTTC | |
| | CCTGACCACGTGGAGCTGAGCTGGTGGGTGAATGG | |
| | GAAGGAGGTGCACAGTGGGGTCAGCACGGACCCGC | |
| | AGCCCCTCAAGGAGCAGCCCGCCCTCAATGACTCCA | |
| | GATACTGCCTGAGCAGCCGCCTGAGGGTCTCGGCCA | |
| | CCTTCTGGCAGAACCCCCGCAACCACTTCCGCTGTC | |
| | AAGTCCAGTTCTACGGGCTCTCGGAGAATGACGAGT | |
| | GGACCCAGGATAGGGCCAAACCCGTCACCCAGATC | |
| | GTCAGCGCCGAGGCCTGGGGTAGAGCAGACTGTGG | |
| | CTTTACCTCGGTGTCCTACCAGCAAGGGGTCCTGTC | |
| | TGCCACCATCCTCTATGAGATCCTGCTAGGGAAGGC | |
| | CACCCTGTATGCTGTGCTGGTCAGCGCCCTTGTGTTG | |
| | ATGGCCATGGTCAAGAGAAAGGATTTCTAA | |
| KIF2C-404 F11 TCR | MGTRLLCWAALCLLGAELTEAGVAQSPRYKIIEKRQS | 11 |
| (TRAV21*02F- | VAFWCNPISGHATLYWYQQILGQGPKLLIQFQNNGVV | |
| TRBV11-2*03F) Protein | DDSQLPKDRFSAERLKGVDSTLKIQPAKLEDSAVYLC | |
| sequence Beta Chain | ASSYHSYGYTFGSGTRLTVVEDLNKVFPPEVAVFEPSE | |
| | AEISHTQKATLVCLATGFFPDHVELSWWVNGKEVHSG | |
| | VSTDPQPLKEQPALNDSRYCLSSRLRVSATFWQNPRN | |
| | HFRCQVQFYGLSENDEWTQDRAKPVTQIVSAEAWGR | |
| | ADCGFTSVSYQQGVLSATILYEILLGKATLYAVLVSAL | |
| | VLMAMVKRKDF | |
| KIF2C-404 F11 TCR | EAGVAQSPRYKIIEKRQSVAFWCNPISGHATLYWYQQI | 12 |
| (TRAV21*02F- | LGQGPKLLIQFQNNGVVDDSQLPKDRFSAERLKGVDS | |
| TRBV11-2*03F) | TLKIQPAKLEDSAVYLCASSYHSYGYTFGSGTRLTVV | |
| Variable region: Beta | | |
| Chain | | |
| KIF2C-404 F11 TCR | MGTRLLCWAALCLLGAELT | 13 |
| (TRAV21*02F- | | |
| TRBV11-2*03F) Leader | | |
| peptide: Beta Chain | | |
| KIF2C-404 F11 TCR | SGHAT | 14 |
| (TRAV21*02F- | | |
| TRBV11-2*03F) Beta | | |
| Chain CDR1 | | |
| KIF2C-404 F11 TCR | FQNNGV | 15 |
| (TRAV21*02F- | | |
| TRBV11-2*03F) Beta | | |
| Chain CDR2 | | |
| KIF2C-404 F11 TCR | ASSYHSYGYT | 16 |
| (TRAV21*02F- | | |
| TRBV11-2*03F) Beta | | |
| Chain CDR3 | | |
| HMD-375 CTL B5 TCR | ATGGCATGCCCTGGCTTCCTGTGGGCACTTGTGATC | 17 |
| (TRAV38-2/DV8*01 F, | TCCACCTGTCTTGAATTTAGCATGGCTCAGACAGTC | |
| TRBV7-9*03 F) DNA | ACTCAGTCTCAACCAGAGATGTCTGTGCAGGAGGCA | |
| sequence: Alpha Chain | GAGACCGTGACCCTGAGCTGCACATATGACACCAGT | |
| | GAGAGTGATTATTATTTATTCTGGTACAAGCAGCCT | |
| | CCCAGCAGGCAGATGATTCTCGTTATTCGCCAAGAA | |
| | GCTTATAAGCAACAGAATGCAACAGAGAATCGTTTC | |
| | TCTGTGAACTTCCAGAAAGCAGCCAAATCCTTCAGT | |
| | CTCAAGATCTCAGACTCACAGCTGGGGGATGCCGCG | |
| | ATGTATTTCTGTGCTTATAGGAGCGCGGTTCGGCAG | |
| | AAGCTGCTCTTTGCAAGGGGAACCATGTTAAAGGTG | |
| | GATCTTAATATCCAGAACCCTGACCCTGCCGTGTAC | |
| | CAGCTGAGAGACTCTAAATCCAGTGACAAGTCTGTC | |
| | TGCCTATTCACCGATTTTGATTCTCAAACAAATGTGT | |
| | CACAAAGTAAGGATTCTGATGTGTATATCACAGACA | |
| | AAACTGTGCTAGACATGAGGTCTATGGACTTCAAGA | |
| | GCAACAGTGCTGTGGCCTGGAGCAACAAATCTGACT | |
| | TTGCATGTGCAAACGCCTTCAACAACAGCATTATTC | |
| | CAGAAGACACCTTCTTCCCCAGCCCAGAAAGTTCCT | |
| | GTGATGTCAAGCTGGTCGAGAAAAGCTTTGAAACA | |
| | GATACGAACCTAAACTTTCAAAACCTGTCAGTGATT | |
| | GGGTTCCGAATCCTCCTCCTGAAAGTGGCCGGGTTT | |
| | AATCTGCTCATGACGCTGCGGCTGTGGTCCAGCTAA | |
| HMD-375 CTL B5 TCR | MACPGFLWALVISTCLEFSMAQTVTQSQPEMSVQEAE | 18 |
| (TRAV38-2/DV8*01 F, | TVTLSCTYDTSESDYYLFWYKQPPSRQMILVIRQEAYK | |
| TRBV7-9*03 F) Protein | QQNATENRFSVNFQKAAKSFSLKISDSQLGDAAMYFC | |
| sequence Alpha Chain | AYRSAVRQKLLFARGTMLKVDLNIQNPDPAVYQLRDS | |
| | KSSDKSVCLFTDFDSQTNVSQSKDSDVYITDKTVLDM | |
| | RSMDFKSNSAVAWSNKSDFACANAFNNSIIPEDTFFPS | |
| | PESSCDVKLVEKSFETDTNLNFQNLSVIGFRILLLKVAG | |
| | FNLLMTLRLWSS | |
| HMD-375 CTL B5 TCR | AQTVTQSQPEMSVQEAETVTLSCTYDTSESDYYLFWY | 19 |
| (TRAV38-2/DV8*01 F, | KQPPSRQMILVIRQEAYKQQNATENRFSVNFQKAAKS | |
| TRBV7-9*03 F) | FSLKISDSQLGDAAMYFCAYRSAVRQKLLFARGTMLK | |
| Variable region: Alpha | VDLN | |
| Chain | | |
| HMD-375 CTL B5 TCR | MACPGFLWALVISTCLEFSM | 20 |
| (TRAV38-2/DV8*01 F, | | |
| TRBV7-9*03 F) Leader | | |
| peptide: Alpha Chain | | |
| HMD-375 CTL B5 TCR | TSESDYY | 21 |
| (TRAV38-2/DV8*01 F, | | |
| TRBV7-9*03 F) Alpha | | |
| Chain CDR1 | | |
| HMD-375 CTL B5 TCR | QEAYKQQN | 22 |
| (TRAV38-2/DV8*01 F, | | |
| TRBV7-9*03 F) Alpha | | |
| Chain CDR2 | | |
| HMD-375 CTL B5 TCR | AYRSAVRQKLL | 23 |
| (TRAV38-2/DV8*01 F, | | |
| TRBV7-9*03 F) Alpha | | |
| Chain CDR3 | | |
| HMD-375 CTL B5 TCR | ATGGGCACCAGCCTCCTCTGCTGGATGGCCCTGTGT | 24 |
| (TRAV38-2/DV8*01 F, | CTCCTGGGGGCAGATCACGCAGATACTGGAGTCTCC | |
| TRBV7-9*03 F) DNA | CAGGACCCCAGACACAAGATCACAAAGAGGGGACA | |
| sequence: Beta Chain | GAATGTAACTTTCAGGTGTGATCCAATTTCTGAACA | |
| | CAACCGCCTTTATTGGTACCGACAGACCCTGGGGCA | |
| | GGGCCCAGAGTTTCTGACTTACTTCCAGAATGAAGC | |
| | TCAACTAGAAAAATCAAGGCTGCTCAGTGATCGGTT | |
| | CTCTGCAGAGAGGCCTAAGGGATCTTTCTCCACCTT | |
| | GGAGATCCAGCGCACAGAGCAGGGGGACTCGGCCA | |
| | TGTATCTCTGTGCCAGCAGCTCCCCGGGGGGCCGGT | |
| | CCTACAATGAGCAGTTCTTCGGGCCAGGGACACGGC | |
| | TCACCGTGCTAGAGGACCTGAAAAACGTGTTCCCAC | |
| | CCGAGGTCGCTGTGTTTGAGCCATCAGAAGCAGAGA | |
| | TCTCCCACACCCAAAAGGCCACACTGGTGTGCCTGG | |
| | CCACAGGCTTCTTCCCTGACCACGTGGAGCTGAGCT | |
| | GGTGGGTGAATGGGAAGGAGGTGCACAGTGGGGTC | |
| | AGCACGGACCCGCAGCCCCTCAAGGAGCAGCCCGC | |
| | CCTCAATGACTCCAGATACTGCCTGAGCAGCCGCCT | |
| | GAGGGTCTCGGCCACCTTCTGGCAGAACCCCCGCAA | |
| | CCACTTCCGCTGTCAAGTCCAGTTCTACGGGCTCTC | |
| | GGAGAATGACGAGTGGACCCAGGATAGGGCCAAAC | |
| | CCGTCACCCAGATCGTCAGCGCCGAGGCCTGGGGTA | |
| | GAGCAGACTGTGGCTTTACCTCGGTGTCCTACCAGC | |
| | AAGGGGTCCTGTCTGCCACCATCCTCTATGAGATCC | |
| | TGCTAGGGAAGGCCACCCTGTATGCTGTGCTGGTCA | |
| | GCGCCCTTGTGTTGATGGCCATGGTCAAGAGAAAGG | |
| | ATTTCTAA | |
| HMD-375 CTL B5 TCR | MGTSLLCWMALCLLGADHADTGVSQDPRHKITKRGQ | 25 |
| (TRAV38-2/DV8*01 F, | NVTFRCDPISEHNRLYWYRQTLGQGPEFLTYFQNEAQ | |
| TRBV7-9*03 F) Protein | LEKSRLLSDRFSAERPKGSFSTLEIQRTEQGDSAMYLC | |
| sequence Beta Chain | ASSSPGGRSYNEQFFGPGTRLTVLEDLKNVFPPEVAVF | |
| | EPSEAEISHTQKATLVCLATGFFPDHVELSWWVNGKE | |
| | VHSGVSTDPQPLKEQPALNDSRYCLSSRLRVSATFWQ | |
| | NPRNHFRCQVQFYGLSENDEWTQDRAKPVTQIVSAEA | |
| | WGRADCGFTSVSYQQGVLSATILYEILLGKATLYAVL | |
| | VSALVLMAMVKRKDF | |
| HMD-375 CTL B5 TCR | DTGVSQDPRHKITKRGQNVTFRCDPISEHNRLYWYRQ | 26 |
| (TRAV38-2/DV8*01 F, | TLGQGPEFLTYFQNEAQLEKSRLLSDRFSAERPKGSFS | |
| TRBV7-9*03 F) | TLEIQRTEQGDSAMYLCASSSPGGRSYNEQFFGPGTRL | |
| Variable region: Beta | TVL | |
| Chain | | |
| HMD-375 CTL B5 TCR | MGTSLLCWMALCLLGADHA | 27 |
| (TRAV38-2/DV8*01 F, | | |
| TRBV7-9*03 F) Leader | | |
| peptide: Beta Chain | | |
| HMD-375 CTL B5 TCR | SEHNR | 28 |
| (TRAV38-2/DV8*01 F, | | |
| TRBV7-9*03 F) Beta | | |
| Chain CDR1 | | |
| HMD-375 CTL B5 TCR | FQNEAQ | 29 |
| (TRAV38-2/DV8*01 F, | | |
| TRBV7-9*03 F) Beta | | |
| Chain CDR2 | | |
| HMD-375 CTL B5 TCR | ASSSPGGRSYNEQF | 30 |
| (TRAV38-2/DV8*01 F, | | |
| TRBV7-9*03 F) Beta | | |
| Chain CDR3 | | |

### III. Cell Penetrating Peptides

A peptide of the disclosure may also be associated with or covalently bound to a cell penetrating peptide (CPP). Cell penetrating peptides that may be covalently bound to a peptide of the disclosure include, e.g., HIV Tat, herpes virus VP22, the Drosophila Antennapedia homeobox gene product, signal sequences, fusion sequences, or protegrin I. Covalently binding a peptide to a CPP can prolong the presentation of a peptide by dendritic cells, thus enhancing antitumour immunity (Wang and Wang, 2002). In some aspects, a peptide of the disclosure (e.g., comprised within a peptide or polyepitope string) may be covalently bound (e.g., via a peptide bond) to a CPP to generate a fusion protein. In other aspects, a peptide or nucleic acid encoding a peptide, according to the current disclosure, may be encapsulated within or associated with a liposome, such as a mulitlamellar, vesicular, or multivesicular liposome.

As used herein, “association” means a physical association, a chemical association or both. For example, an association can involve a covalent bond, a hydrophobic interaction, encapsulation, surface adsorption, or the like.

As used herein, “cell penetrator” refers to a composition or compound which enhances the intracellular delivery of the peptide/polyepitope string to the antigen presenting cell. For example, the cell penetrator may be a lipid which, when associated with the peptide, enhances its capacity to cross the plasma membrane. Alternatively, the cell penetrator may be a peptide. Cell penetrating peptides (CPPs) are known in the art, and include, e.g., the Tat protein of HIV (Frankel and Pabo, 1988), the VP22 protein of HSV (Elliott and O'Hare, 1997) and fibroblast growth factor (Lin et al., 1995).

Cell-penetrating peptides (or “protein transduction domains”) have been identified from the third helix of the Drosophila Antennapedia homeobox gene (Antp), the HIV Tat, and the herpes virus VP22, all of which contain positively charged domains enriched for arginine and lysine residues (Schwarze et al., 2000; Schwarze et al., 1999). Also, hydrophobic peptides derived from signal sequences have been identified as cell-penetrating peptides. (Rojas et al., 1996; Rojas et al., 1998; Du et al., 1998). Coupling these peptides to marker proteins such as β-galactosidase has been shown to confer efficient internalization of the marker protein into cells, and chimeric, in-frame fusion proteins containing these peptides have been used to deliver proteins to a wide spectrum of cell types both in vitro and in vivo (Drin et al., 2002). Fusion of these cell penetrating peptides to a peptide of the disclosure may enhance cellular uptake of the polypeptides.

In some aspects, cellular uptake is facilitated by the attachment of a lipid, such as stearate or myristilate, to the polypeptide. Lipidation has been shown to enhance the passage of peptides into cells. The attachment of a lipid moiety is another way that the present invention increases polypeptide uptake by the cell.

A peptide of the disclosure may be included in a liposomal vaccine composition. For example, the liposomal composition may be or comprise a proteoliposomal composition. Methods for producing proteoliposomal compositions that may be used with the present invention are described, e.g., in Neclapu et al. (2007) and Popescu et al. (2007). In some aspects, protecoliposomal compositions may be used to treat a melanoma.

By enhancing the uptake of a polypeptide of the disclosure, it may be possible to reduce the amount of protein or peptide required for treatment. This in turn can significantly reduce the cost of treatment and increase the supply of therapeutic agent. Lower dosages can also minimize the potential immunogencity of peptides and limit toxic side effects.

In some aspects, a peptide of the disclosure may be associated with a nanoparticle to form nanoparticle-polypeptide complex. In some aspects, the nanoparticle is a liposomes or other lipid-based nanoparticle such as a lipid-based vesicle (e.g., a DOTAP:cholesterol vesicle). In other aspects, the nanoparticle is an iron-oxide based superparamagnetic nanoparticles. Superparamagnetic nanoparticles ranging in diameter from about 10 to 100 nm are small enough to avoid sequestering by the spleen, but large enough to avoid clearance by the liver. Particles this size can penetrate very small capillaries and can be effectively distributed in body tissues. Superparamagnetic nanoparticles-polypeptide complexes can be used as MRI contrast agents to identify and follow those cells that take up the peptide. In some aspects, the nanoparticle is a semiconductor nanocrystal or a semiconductor quantum dot, both of which can be used in optical imaging. In further aspects, the nanoparticle can be a nanoshell, which comprises a gold layer over a core of silica. One advantage of nanoshells is that polypeptides can be conjugated to the gold layer using standard chemistry. In other aspects, the nanoparticle can be a fullerene or a nanotube (Gupta et al., 2005).

Peptides are rapidly removed from the circulation by the kidney and are sensitive to degradation by proteases in serum. By associating a peptide with a nanoparticle, the nanoparticle-polypeptide complexes of the present invention may protect against degradation and/or reduce clearance by the kidney. This may increase the serum half-life of polypeptides, thereby reducing the polypeptide dose need for effective therapy. Further, this may decrease the costs of treatment, and minimizes immunological problems and toxic reactions of therapy.

### IV. Polyepitope Strings

In some aspects, a peptide is included or comprised in a polyepitope string. A polyepitope string is a peptide or polypeptide containing a plurality of antigenic epitopes from one or more antigens linked together. A polyepitope string may be used to induce an immune response in a subject, such as a human subject. Polyepitope strings have been previously used to target malaria and other pathogens (Baraldo et al., 2005; Moorthy et al., 2004; Baird et al., 2004). A polyepitope string may refer to a nucleic acid (e.g., a nucleic acid encoding a plurality of antigens including a peptide of the disclosure) or a peptide or polypeptide (e.g., containing a plurality of antigens including a peptide of the disclosure). A polyepitope string may be included in a cancer vaccine composition.

### V. Applications of Antigenic Peptides

Various aspects are directed to development of and use of antigenic peptides that that are useful for treating and preventing certain cancers. In many aspects, antigenic peptides are produced by chemical synthesis or by molecular expression in a host cell. Peptides can be purified and utilized in a variety of applications including (but not limited to) assays to determine peptide immunogenicity, assays to determine recognition by T cells, peptide vaccines for treatment of cancer, development of modified TCRs of T cells, and development of antibodies.

Peptides can be synthesized chemically by a number of methods. One common method is to use solid-phase peptide synthesis (SPPS). Generally, SPPS is performed by repeating cycles of alternate N-terminal deprotection and coupling reactions, building peptides from the c-terminus to the n-terminus. The c-terminus of the first amino acid is coupled the resin, wherein then the amine is deprecated and then coupled with the free acid of the second amino acid. This cycle repeats until the peptide is synthesized.

Peptides can also be synthesized utilizing molecular tools and a host cell. Nucleic acid sequences corresponding with antigenic peptides can be synthesized. In some aspects, synthetic nucleic acids synthesized in in vitro synthesizers (e.g., phosphoramidite synthesizer), bacterial recombination system, or other suitable methods. Furthermore, synthesized nucleic acids can be purified and lyophilized, or kept stored in a biological system (e.g., bacteria, yeast). For use in a biological system, synthetic nucleic acid molecules can be inserted into a plasmid vector, or similar. A plasmid vector can also be an expression vector, wherein a suitable promoter and a suitable 3′-polyA tail is combined with the transcript sequence.

Aspects are also directed to expression vectors and expression systems that produce antigenic peptides or proteins. These expression systems can incorporate an expression vector to express transcripts and proteins in a suitable expression system. Typical expression systems include bacterial (e.g., E. coli), insect (e.g., SF9), yeast (e.g., S. cerevisiae), animal (e.g., CHO), or human (e.g., HEK 293) cell lines. RNA and/or protein molecules can be purified from these systems using standard biotechnology production procedures.

Assays to determine immunogenicity and/or TCR binding can be performed. One such as is the dextramer flow cytometry assay. Generally, custom-made HLA-matched MHC Class I dextramer:peptide (pMHC) complexes are developed or purchased (Immudex, Copenhagen, Denmark). T cells from peripheral blood mononuclear cells (PBMCs) or tumor-infiltrating lymphocytes (TILs) are incubated the pMHC complexes and stained, which are then run through a flow cytometer to determine if the peptide is capable of binding a TCR of a T cell.

The peptides of the disclosure can also be used to isolate and/or identify T-cell receptors that bind to the peptide. T-cell receptors comprise two different polypeptide chains, termed the T-cell receptor α (TCRα) and B (TCRβ) chains, linked by a disulfide bond. These α:β heterodimers are very similar in structure to the Fab fragment of an immunoglobulin molecule, and they account for antigen recognition by most T cells. A minority of T cells bear an alternative, but structurally similar, receptor made up of a different pair of polypeptide chains designated γ and δ. Both types of T-cell receptor differ from the membrane-bound immunoglobulin that serves as the B-cell receptor: a T-cell receptor has only one antigen-binding site, whereas a B-cell receptor has two, and T-cell receptors are never secreted, whereas immunoglobulin can be secreted as antibody.

Both chains of the T-cell receptor have an amino-terminal variable (V) region with homology to an immunoglobulin V domain, a constant (C) region with homology to an immunoglobulin C domain, and a short hinge region containing a cysteine residue that forms the interchain disulfide bond. Each chain spans the lipid bilayer by a hydrophobic transmembrane domain, and ends in a short cytoplasmic tail.

The three-dimensional structure of the T-cell receptor has been determined. The structure is indeed similar to that of an antibody Fab fragment, as was suspected from earlier studies on the genes that encoded it. The T-cell receptor chains fold in much the same way as those of a Fab fragment, although the final structure appears a little shorter and wider. There are, however, some distinct differences between T-cell receptors and Fab fragments. The most striking difference is in the Cα domain, where the fold is unlike that of any other immunoglobulin-like domain. The half of the domain that is juxtaposed with the CB domain forms a B sheet similar to that found in other immunoglobulin-like domains, but the other half of the domain is formed of loosely packed strands and a short segment of a helix. The intramolecular disulfide bond, which in immunoglobulin-like domains normally joins two β strands, in a Cα domain joins a β strand to this segment of a helix.

There are also differences in the way in which the domains interact. The interface between the V and C domains of both T-cell receptor chains is more extensive than in antibodies, which may make the hinge joint between the domains less flexible. And the interaction between the Cα and CB domains is distinctive in being assisted by carbohydrate, with a sugar group from the Cα domain making a number of hydrogen bonds to the CB domain. Finally, a comparison of the variable binding sites shows that, although the complementarity-determining region (CDR) loops align fairly closely with those of antibody molecules, there is some displacement relative to those of the antibody molecule. This displacement is particularly marked in the Vα CDR2 loop, which is oriented at roughly right angles to the equivalent loop in antibody V domains, as a result of a shift in the B strand that anchors one end of the loop from one face of the domain to the other. A strand displacement also causes a change in the orientation of the Vβ CDR2 loop in two of the seven Vβ domains whose structures are known. As yet, the crystallographic structures of seven T-cell receptors have been solved to this level of resolution.

Aspects of the disclosure relate to engineered T cell receptors that bind a peptide of the disclosure, such as a peptide of SEQ ID NO: 1 or 2. The term “engineered” refers to T cell receptors that have TCR variable regions grafted onto TCR constant regions to make a chimeric polypeptide that binds to peptides and antigens of the disclosure. In certain aspects, the TCR comprises intervening sequences that are used for cloning, enhanced expression, detection, or for therapeutic control of the construct, but are not present in endogenous TCRs, such as multiple cloning sites, linker, hinge sequences, modified hinge sequences, modified transmembrane sequences, a detection polypeptide or molecule, or therapeutic controls that may allow for selection or screening of cells comprising the TCR.

In some aspects, the TCR comprises non-TCR sequences. Accordingly, certain aspects relate to TCRs with sequences that are not from a TCR gene. In some aspects, the TCR is chimeric, in that it contains sequences normally found in a TCR gene, but contains sequences from at least two TCR genes that are not necessarily found together in nature.

### VI. Antibodies

Aspects of the disclosure relate to antibodies that target the peptides of the disclosure, or fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (u), delta (8), gamma (Y), alpha (a), or epsilon (8) chains, respectively. IgG has several subtypes, including, but not limited to, IgG1, IgG2, IgG3, and IgG4. IgM subtypes include IgM1 and IgM2. IgA subtypes include IgA1 and IgA2.

### VII. Antibody Conjugates

Aspects of the disclosure relate to antibodies against a peptide of the disclosure, generally of the monoclonal type, that are linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radio-labeled nucleotides, antiviral agents, chelating agents, cytokines, growth factors, and oligo- or poly-nucleotides. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Any antibody of sufficient selectivity, specificity or affinity may be employed as the basis for an antibody conjugate. Such properties may be evaluated using conventional immunological screening methodology known to those of skill in the art. Sites for binding to biological active molecules in the antibody molecule, in addition to the canonical antigen binding sites, include sites that reside in the variable domain that can bind pathogens, B-cell superantigens, the T cell co-receptor CD4 and the HIV-1 envelope (Sasso et al., 1989; Shorki et al., 1991; Silvermann et al., 1995; Cleary et al., 1994; Lenert et al., 1990; Berberian et al., 1993; Kreier et al., 1991). In addition, the variable domain is involved in antibody self-binding (Kang et al., 1988), and contains epitopes (idiotopes) recognized by anti-antibodies (Kohler et al., 1989).

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired. Another such example is the formation of a conjugate comprising an antibody linked to a cytotoxic or anti-cellular agent, and may be termed “immunotoxins”.

Antibody conjugates are generally preferred for use as diagnostic agents. Antibody diagnostics generally fall within two classes, those for use in in vitro diagnostics, such as in a variety of immunoassays, and/or those for use in vivo diagnostic protocols, generally known as “antibody-directed imaging”.

Many appropriate imaging agents are known in the art, as are methods for their attachment to antibodies (see, for e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; X-ray imaging.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸ cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium ¹⁸⁶, rhenium¹⁸⁸, ⁷⁵selenium, ³⁵sulphur, technicium^{99m} and/or yttrium⁹⁰. ¹²⁵I is often being preferred for use in certain aspects, and technicium^{99m} and/or indium¹¹¹ are also often preferred due to their low energy and suitability for long range detection. Radioactively labeled monoclonal antibodies of the present invention may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the invention may be labeled with technetium99m by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNC12, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodaminc Red, Renographin, ROX, TAMRA, TET, Tetramethylrhodamine, and/or Texas Red.

Another type of antibody conjugates contemplated in the present invention are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other aspects, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

In another aspect of the disclosure, the antibody may be linked to semiconductor nanocrystals such as those described in U.S. Pat. Nos. 6,048,616; 5,990,479; 5,690,807; 5,505,928; 5,262,357 (all of which are incorporated herein in their entireties); as well as PCT Publication No. 99/26299 (published May 27, 1999). In particular, exemplary materials for use as semiconductor nanocrystals in the biological and chemical assays of the present invention include, but are not limited to those described above, including group II-VI, III-V and group IV semiconductors such as ZnS, ZnSe, ZnTe, CdS, CdSe, CdTe, MgS, MgSe, MgTe, CaS, CaSe, CaTe, SrS, SrSe, SrTe, BaS, BaSe, BaTe, GaN, GaP, GaAs, GaSb, InP, InAs, InSb, AIS, AlP, AlSb, PbS, PbSe, Ge and Si and ternary and quaternary mixtures thereof. Methods for linking semiconductor nanocrystals to antibodies are described in U.S. Pat. Nos. 6,630,307 and 6,274,323.

In still further aspects, the present invention concerns immunodetection methods for binding, purifying, removing, quantifying and/or otherwise generally detecting biological components such as T cells or that selectively bind or recognize a peptide of the disclosure. In some aspects, a tetramer assay may be used with the present invention. Tetramer assays generally involve generating soluble peptide-MHC tetramers that may bind antigen specific T lymphocytes, and methods for tetramer assays are described, e.g., in Altman et al. (1996). Some immunodetection methods that may be used include, e.g., enzyme linked immunosorbent assay (ELISA), radioimmunoassay (RIA), immunoradiometric assay, fluoroimmunoassay, chemiluminescent assay, bioluminescent assay, tetramer assay, and Western blot. The steps of various useful immunodetection methods have been described in the scientific literature, such as, e.g., Doolittle and Ben-Zeev, 1999; Gulbis and Galand, 1993; De Jager et al., 1993; and Nakamura et al., 1987, each incorporated herein by reference.

### VIII. MHC Polypeptides

Aspects of the disclosure relate to compositions comprising MHC polypeptides. In some aspects, the MHC polypeptide comprises at least 2, 3, or 4 MHC polypeptides that may be expressed as separate polypeptides or as a fusion protein. Presentation of antigens to T cells is mediated by two distinct classes of molecules MHC class I (MHC-I) and MHC class II (MHC-II) (also identified as “pMHC” herein), which utilize distinct antigen processing pathways. Peptides derived from intracellular antigens are presented to CD8+ T cells by MHC class I molecules, which are expressed on virtually all cells, while extracellular antigen-derived peptides are presented to CD4+ T cells by MHC-II molecules. In certain aspects, a particular antigen is identified and presented in the antigen-MHC complex in the context of an appropriate MHC class I or II polypeptide. In certain aspects, the genetic makeup of a subject may be assessed to determine which MHC polypeptide is to be used for a particular patient and a particular set of peptides. In certain aspects, the MHC class 1 polypeptide comprises all or part of a HLA-A. HLA-B, HLA-C, HLA-E, HLA-F. HLA-G or CD-1 molecule. In aspects wherein the MHC polypeptide is a MHC class II polypeptide, the MHC class II polypeptide can comprise all or a part of a HLA-DR, HLA-DQ, or HLA-DP.

Non-classical MHC polypeptides are also contemplated for use in MHC complexes of the invention. Non-classical MHC polypeptides are non-polymorphic, conserved among species, and possess narrow, deep, hydrophobic ligand binding pockets. These binding pockets are capable of presenting glycolipids and phospholipids to Natural Killer T (NKT) cells or certain subsets of CD8+ T-cells such as Qa1, HLA-E-restricted CD8+ T-cells, or MAIT cells. NKT cells represent a unique lymphocyte population that co-express NK cell markers and a semi-invariant T cell receptor (TCR). They are implicated in the regulation of immune responses associated with a broad range of diseases.

### IX. Host Cells

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In certain aspects transfection can be carried out on any prokaryotic or eukaryotic cell. In some aspects electroporation involves transfection of a human cell. In other aspects electroporation involves transfection of an animal cell. In certain aspects transfection involves transfection of a cell line or a hybrid cell type. In some aspects the cell or cells being transfected are cancer cells, tumor cells or immortalized cells. In some instances tumor, cancer, immortalized cells or cell lines are induced and in other instances tumor, cancer, immortalized cells or cell lines enter their respective state or condition naturally. In certain aspects the cells or cell lines can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH3T3, NIH3T3L1, K562, NK-cells, NS0, Panc-1, PC12, PC-3, Peripheral blood cells, Plasma cells, Primary Fibroblasts, RBL, Renca, RLE, SF21, SF9, SH-SY5Y, SK-MES-1, SK-N-SH, SL3, SW403, Stimulus-triggered Acquisition of Pluripotency (STAP) cell or derivate SW403, T-cells, THP-1, Tumor cells, U2OS, U937, peripheral blood lymphocytes, expanded T cells, hematopoietic stem cells, or Vero cells.

### X. Additional Agents

### A. Immunostimulators

In some aspects, the method further comprises administration of an additional agent. In some aspects, the additional agent is an immunostimulator. The term “immunostimulator” as used herein refers to a compound that can stimulate an immune response in a subject, and may include an adjuvant. In some aspects, an immunostimulator is an agent that does not constitute a specific antigen, but can boost the strength and longevity of an immune response to an antigen. Such immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherichia coli, Salmonella minnesota, Salmonella typhimurium, or Shigella flexneri or specifically with MPL (ASO4), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments.

In some aspects, the additional agent comprises an agonist for pattern recognition receptors (PRR), including, but not limited to Toll-Like Receptors (TLRs), specifically TLRs 2, 3, 4, 5, 7, 8, 9 and/or combinations thereof. In some aspects, additional agents comprise agonists for Toll-Like Receptors 3, agonists for Toll-Like Receptors 7 and 8, or agonists for Toll-Like Receptor 9; preferably the recited immunostimulators comprise imidazoquinolines; such as R848; adenine derivatives, such as those disclosed in U.S. Pat. No. 6,329,381, U.S. Published Patent Application 2010/0075995, or WO 2010/018132; immunostimulatory DNA; or immunostimulatory RNA. In some aspects, the additional agents also may comprise immunostimulatory RNA molecules, such as but not limited to dsRNA, poly I:C or poly I:poly C12U (available as Ampligen®, both poly I:C and poly I:polyC12U being known as TLR3 stimulants), and/or those disclosed in F. Heil et al., “Species-Specific Recognition of Single-Stranded RNA via Toll-like Receptor 7 and 8” Science 303(5663), 1526-1529 (2004); J. Vollmer et al., “Immune modulation by chemically modified ribonucleosides and oligoribonucleotides” WO 2008033432 A2; A. Forsbach et al., “Immunostimulatory oligoribonucleotides containing specific sequence motif(s) and targeting the Toll-like receptor 8 pathway” WO 2007062107 A2; E. Uhlmann et al., “Modified oligoribonucleotide analogs with enhanced immunostimulatory activity” U.S. Pat. Appl. Publ. US 2006241076; G. Lipford et al., “Immunostimulatory viral RNA oligonucleotides and use for treating cancer and infections” WO 2005097993 A2; G. Lipford et al., “Immunostimulatory G.U-containing oligoribonucleotides, compositions, and screening methods” WO 2003086280 A2. In some aspects, an additional agent may be a TLR-4 agonist, such as bacterial lipopolysaccharide (LPS), VSV-G, and/or HMGB-1. In some aspects, additional agents may comprise TLR-5 agonists, such as flagellin, or portions or derivatives thereof, including but not limited to those disclosed in U.S. Pat. Nos. 6,130,082, 6,585,980, and 7,192,725.

In some aspects, additional agents may be proinflammatory stimuli released from necrotic cells (e.g., urate crystals). In some aspects, additional agents may be activated components of the complement cascade (e.g., CD21, CD35, etc.). In some aspects, additional agents may be activated components of immune complexes. Additional agents also include complement receptor agonists, such as a molecule that binds to CD21 or CD35. In some aspects, the complement receptor agonist induces endogenous complement opsonization of the synthetic nanocarrier. In some aspects, immunostimulators are cytokines, which are small proteins or biological factors (in the range of 5 kD-20 kD) that are released by cells and have specific effects on cell-cell interaction, communication and behavior of other cells. In some aspects, the cytokine receptor agonist is a small molecule, antibody, fusion protein, or aptamer.

### B. Immunotherapies

In some aspects, the additional therapy comprises a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies are known in the art, and some are described below.

### 1. Inhibition of Co-Stimulatory Molecules

In some aspects, the immunotherapy comprises an inhibitor of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 2. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7. TLR8 or CD40 have been used as antibody targets.

### 3. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some aspects, the CAR-T therapy targets CD19.

### 4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in antiviral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 5. Adoptive T-Cell Therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

### 6. Checkpoint Inhibitors and Combination Treatment

In some aspects, the additional therapy comprises immune checkpoint inhibitors. Certain aspects are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some aspects, PD-1, PDL1, and PDL2 are human PD-1, PDL1 and PDL2.

In some aspects, the PD-1 inhibitor is a molecule that inhibits the binding of PD-1 to its ligand binding partners. In a specific aspect, the PD-1 ligand binding partners are PDL1 and/or PDL2. In another aspect, a PDL1 inhibitor is a molecule that inhibits the binding of PDL1 to its binding partners. In a specific aspect, PDL1 binding partners are PD-1 and/or B7-1. In another aspect, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some aspects, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PDL1, or PDL2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (sec, e.g., WO01/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### C. Oncolytic Virus

In some aspects, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumour. Oncolytic viruses are thought not only to cause direct destruction of the tumour cells, but also to stimulate host anti-tumour immune responses for long-term immunotherapy

### D. Polysaccharides

In some aspects, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### E. Neoantigens

In some aspects, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### F Chemotherapies

In some aspects, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain aspects. In some aspects, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### G. Radiotherapy

In some aspects, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some aspects, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or exactly 1, 2, 3, 4, 5, 6, 7, 8, 9, 10, 11, or 12 (or any derivable range therein) fractionated doses are administered per day. In some aspects, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### H. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### I. Other Agents

It is contemplated that other agents may be used in combination with certain aspects of the present aspects to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other aspects, cytostatic or differentiation agents can be used in combination with certain aspects of the present aspects to improve the anti-hyperproliferative efficacy of the treatments. Inhibitors of cell adhesion are contemplated to improve the efficacy of the present aspects. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present aspects to improve the treatment efficacy.

### XI. Proteinaceous Compositions

As used herein, a “protein” “peptide” or “polypeptide” refers to a molecule comprising at least five amino acid residues. As used herein, the term “wild-type” refers to the endogenous version of a molecule that occurs naturally in an organism. In some aspects, wild-type versions of a protein or polypeptide are employed, however, in many aspects of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some aspects, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular aspects, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain aspects the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 1000, 1200, 1400, 1600, 1800, or 2000 amino acid residues or nucleic acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.).

The polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS:1-34. In specific aspects, the peptide or polypeptide is or is based on a human sequence. In certain aspects, the peptide or polypeptide is not naturally occurring and/or is in a combination of peptides or polypeptides.

In some aspects, the protein, polypeptide, or nucleic acid may comprise amino acids or nucleotides 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) of SEQ ID NOS: 1-34.

In some aspects, the protein may comprise, polypeptide may comprise, nucleic acid may comprise, or polypeptide or protein encoding by a nucleic acid may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids or nucleic acids of SEQ ID NOS: 1-34.

In some aspects, the protein may comprise, polypeptide may comprise, nucleic acid may comprise, or polypeptide or protein encoding by a nucleic acid may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, or 320 (or any derivable range therein) contiguous amino acids of SEQ ID NOS: 1-34 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous to one of SEQ ID NOS:1-34.

In some aspects there is a nucleic acid molecule or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 of any of SEQ ID NOS: 1-34 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, or 950 (or any derivable range therein) contiguous amino acids or nucleotides of any of SEQ ID NOS:1-34.

In some aspects, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the peptide or polypeptide of one of SEQ ID NOS:1-34 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type (or any range derivable therein). A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further aspects, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain aspects, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain aspects, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain aspects, the substitution of amino acids whose hydrophilicity values are within ±2 are included, in other aspects, those which are within ±1 are included, and in still other aspects, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein structure.

In some aspects of the invention, amino acid substitutions are made that: (1) reduce susceptibility to proteolysis, (2) reduce susceptibility to oxidation, (3) alter binding affinity for forming protein complexes. (4) alter ligand or antigen binding affinities, and/or (5) confer or modify other physicochemical or functional properties on such polypeptides. For example, single or multiple amino acid substitutions (in certain aspects, conservative amino acid substitutions) may be made in the naturally occurring sequence. Substitutions can be made in that portion of the antibody that lies outside the domain(s) forming intermolecular contacts. In such aspects, conservative amino acid substitutions can be used that do not substantially change the structural characteristics of the protein or polypeptide (e.g., one or more replacement amino acids that do not disrupt the secondary structure that characterizes the native antibody).

### XII. Nucleic Acids

In certain aspects, nucleic acid sequences can exist in a variety of instances such as: isolated segments and recombinant vectors of incorporated sequences or recombinant polynucleotides encoding peptides and polypeptides of the disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene.” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain aspects, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the case of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Hybridization

The nucleic acids that hybridize to other nucleic acids under particular hybridization conditions. Methods for hybridizing nucleic acids are well known in the art. See, e.g., Current Protocols in Molecular Biology, John Wiley and Sons, N.Y. (1989), 6.3.1-6.3.6. As defined herein, a moderately stringent hybridization condition uses a prewashing solution containing 5× sodium chloride/sodium citrate (SSC), 0.5% SDS, 1.0 mM EDTA (pH 8.0), hybridization buffer of about 50% formamide, 6×SSC, and a hybridization temperature of 55° C. (or other similar hybridization solutions, such as one containing about 50% formamide, with a hybridization temperature of 42° C.), and washing conditions of 60° C. in 0.5×SSC, 0.1% SDS. A stringent hybridization condition hybridizes in 6×SSC at 45° C., followed by one or more washes in 0.1×SSC, 0.2% SDS at 68° C. Furthermore, one of skill in the art can manipulate the hybridization and/or washing conditions to increase or decrease the stringency of hybridization such that nucleic acids comprising nucleotide sequence that are at least 65%, at least 70%, at least 75%, at least 80%, at least 85%, at least 90%, at least 95%, at least 96%, at least 97%, at least 98% or at least 99% identical to each other typically remain hybridized to each other.

The parameters affecting the choice of hybridization conditions and guidance for devising suitable conditions are set forth by, for example, Sambrook, Fritsch, and Maniatis (Molecular Cloning: A Laboratory Manual, Cold Spring Harbor Laboratory Press, Cold Spring Harbor, N.Y., chapters 9 and 11 (1989); Current Protocols in Molecular Biology, Ausubel et al., eds., John Wiley and Sons, Inc., sections 2.10 and 6.3-6.4 (1995), both of which are herein incorporated by reference in their entirety for all purposes) and can be readily determined by those having ordinary skill in the art based on, for example, the length and/or base composition of the DNA.

### B. Mutation

Changes can be introduced by mutation into a nucleic acid, thereby leading to changes in the amino acid sequence of a polypeptide (e.g., an antigenic peptide or polypeptide) that it encodes. Mutations can be introduced using any technique known in the art. In one aspect, one or more particular amino acid residues are changed using, for example, a site-directed mutagenesis protocol. In another aspect, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### C. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another aspect, the nucleic acid molecules may be used as probes or PCR primers for specific nucleic acid sequences. For instance, a nucleic acid molecule probe may be used in diagnostic methods or a nucleic acid molecule PCR primer may be used to amplify regions of DNA that could be used, inter alia, to isolate nucleic acid sequences for use in producing the engineered cells of the disclosure. In a preferred aspect, the nucleic acid molecules are oligonucleotides.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### XIII. Polypeptide Expression

In some aspects, there are nucleic acid molecule encoding polypeptides or peptides of the disclosure (e.g antibodies, TCR genes, MHC molecules, and immunogenic peptides). These may be generated by methods known in the art, e.g., isolated from B cells of mice that have been immunized and isolated, phage display, expressed in any suitable recombinant expression system and allowed to assemble to form antibody molecules or by recombinant methods.

The nucleic acid molecules may be used to express large quantities of polypeptides. If the nucleic acid molecules are derived from a non-human, non-transgenic animal, the nucleic acid molecules may be used for humanization of the antibody or TCR genes.

### A. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, antigenic peptides and polypeptides, TCR genes, MHC molecules, modified antibodies, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### B. Expression Systems

Numerous expression systems exist that comprise at least a part or all of the expression vectors discussed above. Prokaryote- and/or eukaryote-based systems can be employed for use with an aspect to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirullch et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### D. Host Cells

In another aspect, contemplated are the use of host cells into which a recombinant expression vector has been introduced. Polypeptides can be expressed in a variety of cell types. An expression construct encoding a polypeptide or peptide of the disclosure can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### XIV. Formulations and Culture of the Cells

In particular aspects, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V. X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain aspects, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific aspects, one or more of these may be explicitly excluded.

In some aspects, the medium further comprises vitamins. In some aspects, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some aspects, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some aspects, the vitamins include or consist essentially of biotin, DL alpha tocopherol acetate, DL alpha-tocopherol, vitamin A, or combinations or salts thereof. In some aspects, the medium further comprises proteins. In some aspects, the proteins comprise albumin or bovine serum albumin, a fraction of BSA, catalase, insulin, transferrin, superoxide dismutase, or combinations thereof. In some aspects, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some aspects, the medium comprises one or more of the following: a B-27® supplement, xeno-free B-27® supplement, GS21TM supplement, or combinations thereof. In some aspects, the medium comprises or further comprises amino acids, monosaccharides, inorganic ions. In some aspects, the amino acids comprise arginine, cystine, isoleucine, leucine, lysine, methionine, glutamine, phenylalanine, threonine, tryptophan, histidine, tyrosine, or valine, or combinations thereof. In some aspects, the inorganic ions comprise sodium, potassium, calcium, magnesium, nitrogen, or phosphorus, or combinations or salts thereof. In some aspects, the medium further comprises one or more of the following: molybdenum, vanadium, iron, zinc, selenium, copper, or manganese, or combinations thereof. In certain aspects, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific aspects, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific aspects, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific aspects, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular aspects the cells are in a formulated cell suspension that is stable at room temperature for 1, 2, 3, or 4 hours or more from time of thawing.

In some aspects, the method further comprises priming the T cells. In some aspects, the T cells are primed with antigen presenting cells. In some aspects, the antigen presenting cells present tumor antigens or peptides, such as those disclosed herein.

In particular aspects, the cells of the disclosure comprise an exogenous TCR, which may be of a defined antigen specificity, such as defined antigen specificity to SEQ ID NO:1. In some aspects, the TCR can be selected based on absent or reduced alloreactivity to the intended recipient (examples include certain virus-specific TCRs, xeno-specific TCRs, or cancer-testis antigen-specific TCRs). In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some aspects, the choice of exogenous TCR may not necessarily be defined based on lack of alloreactivity. In some aspects, the endogenous TCR genes have been modified by genome editing so that they do not express a protein. Methods of gene editing such as methods using the CRISPR/Cas9 system are known in the art and described herein.

### XV. Administration of Therapeutic Compositions

Methods of the disclosure relate to the treatment of subjects with cancer. In some aspects, the treatment may be directed to those that have or have been determined to have a cancer for a particular peptide of the disclosure, such as a peptide of SEQ ID NO:1 or 2. In some aspects, the methods may be employed with respect to individuals who have tested positive for such cancer, who have one or more symptoms of a cancer, or who are deemed to be at risk for developing such a cancer.

Certain aspects of the disclosure relate to the treatment of cancer and/or use of cancer antigens. The cancer to be treated or antigen may be an antigen associated with any cancer known in the art or, for example, epithelial cancer, (e.g., breast, gastrointestinal, lung), prostate cancer, bladder cancer, lung (e.g., small cell lung) cancer, colon cancer, ovarian cancer, brain cancer, gastric cancer, renal cell carcinoma, pancreatic cancer, liver cancer, esophageal cancer, head and neck cancer, or a colorectal cancer. In some aspects, the cancer to be treated or antigen is from one of the following cancers: adenocortical carcinoma, agnogenic myeloid metaplasia, AIDS-related cancers (e.g., AIDS-related lymphoma), anal cancer, appendix cancer, astrocytoma (e.g., cerebellar and cerebral), basal cell carcinoma, bile duct cancer (e.g., extrahepatic), bladder cancer, bone cancer, (osteosarcoma and malignant fibrous histiocytoma), brain tumor (e.g., glioma, brain stem glioma, cerebellar or cerebral astrocytoma (e.g., pilocytic astrocytoma, diffuse astrocytoma, anaplastic (malignant) astrocytoma), malignant glioma, ependymoma, oligodenglioma, meningioma, meningiosarcoma, craniopharyngioma, hemangioblastomas, medulloblastoma, supratentorial primitive neuroectodermal tumors, visual pathway and hypothalamic glioma, and glioblastoma), breast cancer, bronchial adenomas/carcinoids, carcinoid tumor (e.g., gastrointestinal carcinoid tumor), carcinoma of unknown primary, central nervous system lymphoma, cervical cancer, colon cancer, colorectal cancer, chronic myeloproliferative disorders, endometrial cancer (e.g., uterine cancer), ependymoma, esophageal cancer, Ewing's family of tumors, eye cancer (e.g., intraocular melanoma and retinoblastoma), gallbladder cancer, gastric (stomach) cancer, gastrointestinal carcinoid tumor, gastrointestinal stromal tumor (GIST), germ cell tumor, (e.g., extracranial, extragonadal, ovarian), gestational trophoblastic tumor, head and neck cancer, hepatocellular (liver) cancer (e.g., hepatic carcinoma and heptoma), hypopharyngeal cancer, islet cell carcinoma (endocrine pancreas), laryngeal cancer, laryngeal cancer, leukemia, lip and oral cavity cancer, oral cancer, liver cancer, lung cancer (e.g., small cell lung cancer, non-small cell lung cancer, adenocarcinoma of the lung, and squamous carcinoma of the lung), lymphoid neoplasm (e.g., lymphoma), medulloblastoma, ovarian cancer, mesothelioma, metastatic squamous neck cancer, mouth cancer, multiple endocrine neoplasia syndrome, myelodysplastic syndromes, myelodysplastic/myeloproliferative diseases, nasal cavity and paranasal sinus cancer, nasopharyngeal cancer, neuroblastoma, neuroendocrine cancer, oropharyngeal cancer, ovarian cancer (e.g., ovarian epithelial cancer, ovarian germ cell tumor, ovarian low malignant potential tumor), pancreatic cancer, parathyroid cancer, penile cancer, cancer of the peritoneal, pharyngeal cancer, pheochromocytoma, pineoblastoma and supratentorial primitive neuroectodermal tumors, pituitary tumor, pleuropulmonary blastoma, lymphoma, primary central nervous system lymphoma (microglioma), pulmonary lymphangiomyomatosis, rectal cancer, renal cancer, renal pelvis and ureter cancer (transitional cell cancer), rhabdomyosarcoma, salivary gland cancer, skin cancer (e.g., non-melanoma (e.g., squamous cell carcinoma), melanoma, and Merkel cell carcinoma), small intestine cancer, squamous cell cancer, testicular cancer, throat cancer, thymoma and thymic carcinoma, thyroid cancer, tuberous sclerosis, urethral cancer, vaginal cancer, vulvar cancer, Wilms' tumor, and post-transplant lymphoproliferative disorder (PTLD), abnormal vascular proliferation associated with phakomatoses, edema (such as that associated with brain tumors), or Meigs' syndrome.

The therapy provided herein may comprise administration of a combination of therapeutic agents, such as a first anti-cancer therapy and a second anti-cancer therapy. The therapies may be administered in any suitable manner known in the art. For example, the first and second cancer treatment may be administered sequentially (at different times) or concurrently (at the same time). In some aspects, the first and second cancer treatments are administered in a separate composition. In some aspects, the first and second cancer treatments are in the same composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some aspects, the cancer therapy is administered intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

The quantity to be administered, both according to number of treatments and unit dose, depends on the treatment effect desired. An effective dose is understood to refer to an amount necessary to achieve a particular effect. In the practice in certain aspects, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain aspects, the effective dose of the pharmaceutical composition is one which can provide a blood level of about 1 μM to 150 μM. In another aspect, the effective dose provides a blood level of about 4 μM to 100 μM; or about 1 μM to 100 μM; or about 1 μM to 50 M; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 M; or about 25 μM to 100 μM; or about 25 μM to 50 μM; or about 50 μM to 150 μM; or about 50 μM to 100 μM (or any range derivable therein). In other aspects, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 UM or any range derivable therein. In certain aspects, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In select aspects, it is contemplated that a peptide of the disclosure may be comprised in a vaccine composition and administered to a subject to induce a therapeutic immune response in the subject towards a cancer. A vaccine composition for pharmaceutical use in a subject may comprise a peptide composition disclosed herein and a pharmaceutically acceptable carrier.

The phrases “pharmaceutical,” “pharmaceutically acceptable,” or “pharmacologically acceptable” refers to molecular entities and compositions that do not produce an adverse, allergic or other untoward reaction when administered to an animal, such as, for example, a human, as appropriate. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington: The Science and Practice of Pharmacy, 21st edition, Pharmaceutical Press, 2011, incorporated herein by reference). Except insofar as any conventional carrier is incompatible with the active ingredient, its use in the vaccine compositions of the present invention is contemplated.

As used herein, a “protective immune response” refers to a response by the immune system of a mammalian host to a cancer. A protective immune response may provide a therapeutic effect for the treatment of a cancer, e.g., decreasing tumor size, increasing survival, etc.

In some aspects, the vaccine composition may be administered by microstructured transdermal or ballistic particulate delivery. Microstructures as carriers for vaccine formulation are a desirable configuration for vaccine applications and are widely known in the art (Gerstel and Place 1976 (U.S. Pat. No. 3,964,482); Ganderton and McAinsh 1974 (U.S. Pat. No. 3,814,097); U.S. Pat. Nos. 5,797,898, 5,770,219 and 5,783,208, and U.S. Patent Application 2005/0065463). Such a vaccine composition formulated for ballistic particulate delivery may comprise an isolated peptide disclosed herein immobilized on a surface of a support substrate. In these aspects, a support substrate can include, but is not limited to, a microcapsule, a microparticle, a microsphere, a nanocapsule, a nanoparticle, a nanosphere, or a combination thereof.

In other aspects, a vaccine composition comprises an immobilized or encapsulated peptide or antibody as disclosed herein and a support substrate. In these aspects, a support substrate can include, but is not limited to, a lipid microsphere, a lipid nanoparticle, an ethosome, a liposome, a niosome, a phospholipid, a sphingosome, a surfactant, a transferosome, an emulsion, or a combination thereof. The formation and use of liposomes and other lipid nano- and microcarrier formulations is generally known to those of ordinary skill in the art, and the use of liposomes, microparticles, nanocapsules and the like have gained widespread use in delivery of therapeutics (e.g., U.S. Pat. No. 5,741,516, specifically incorporated herein in its entirety by reference). Numerous methods of liposome and liposome-like preparations as potential drug carriers, including encapsulation of peptides, have been reviewed (U.S. Pat. Nos. 5,567,434; 5,552,157; 5,565,213; 5,738,868 and 5,795,587, each of which is specifically incorporated in its entirety by reference).

In addition to the methods of delivery described herein, a number of alternative techniques are also contemplated for administering the disclosed vaccine compositions. By way of nonlimiting example, a vaccine composition may be administered by sonophoresis (i.e., ultrasound) which has been used and described in U.S. Pat. No. 5,656,016 for enhancing the rate and efficacy of drug permeation into and through the circulatory system; intraosseous injection (U.S. Pat. No. 5,779,708), or feedback-controlled delivery (U.S. Pat. No. 5,697,899), and each of the patents in this paragraph is specifically incorporated herein in its entirety by reference.

### XVI. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In certain aspects the sample is obtained from a biopsy from ovarian or endometrial tissue by any of the biopsy methods previously mentioned. In other aspects the sample may be obtained from any of the tissues provided herein that include but are not limited to non-cancerous or cancerous tissue and non-cancerous or cancerous tissue from the ovarian epithelium, fallopian epithelium, ovaries, cervix, fallopian tube, or uterus. Alternatively, the sample may be obtained from any other source including but not limited to blood, serum, plasma, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the check, saliva collection, urine collection, feces collection, collection of menses, tears, or semen.

The sample may be obtained by methods known in the art. In certain aspects the samples are obtained by biopsy. In other aspects the sample is obtained by swabbing, endoscopy, scraping, phlebotomy, or any other methods known in the art. In some cases, the sample may be obtained, stored, or transported using components of a kit of the present methods. In some cases, multiple samples, such as multiple plasma or serum samples may be obtained for diagnosis by the methods described herein. In other cases, multiple samples, such as one or more samples from one tissue type (for example ovaries or related tissues) and one or more samples from another specimen (for example serum) may be obtained for diagnosis by the methods. Samples may be obtained at different times are stored and/or analyzed by different methods. For example, a sample may be obtained and analyzed by routine staining methods or any other cytological analysis methods.

In some aspects the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, blood draw, endoscopy, or phlebotomy. The method of needle aspiration may further include fine needle aspiration, core needle biopsy, vacuum assisted biopsy, or large core biopsy. In some aspects, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods.

In some aspects of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some aspects of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some aspects, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### XVII. Detection and Vaccination Kits

A peptide or antibody of the disclosure may be included in a kit. The peptide or antibody in the kit may be detectably labeled or immobilized on a surface of a support substrate also comprised in the kit. The peptide(s) or antibody may, for example, be provided in the kit in a suitable form, such as sterile, lyophilized, or both.

The support substrate comprised in a kit of the invention may be selected based on the method to be performed. By way of nonlimiting example, a support substrate may be a multi-well plate or microplate, a membrane, a filter, a paper, an emulsion, a bead, a microbead, a microsphere, a nanobead, a nanosphere, a nanoparticle, an ethosome, a liposome, a niosome, a transferosome, a dipstick, a card, a celluloid strip, a glass slide, a microslide, a biosensor, a lateral flow apparatus, a microchip, a comb, a silica particle, a magnetic particle, or a self-assembling monolayer.

As appropriate to the method being performed, a kit may further comprise one or more apparatuses for delivery of a composition to a subject or for otherwise handling a composition of the invention. By way of nonlimiting example, a kit may include an apparatus that is a syringe, an eye dropper, a ballistic particle applicator (e.g., applicators disclosed in U.S. Pat. Nos. 5,797,898, 5,770,219 and 5,783,208, and U.S. Patent Application 2005/0065463), a scoopula, a microslide cover, a test strip holder or cover, and such like.

A detection reagent for labeling a component of the kit may optionally be comprised in a kit for performing a method of the present invention. In particular aspects, the labeling or detection reagent is selected from a group comprising reagents used commonly in the art and including, without limitation, radioactive elements, enzymes, molecules which absorb light in the UV range, and fluorophores such as fluorescein, rhodamine, auramine, Texas Red, AMCA blue and Lucifer Yellow. In other aspects, a kit is provided comprising one or more container means and a BST protein agent already labeled with a detection reagent selected from a group comprising a radioactive element, an enzyme, a molecule which absorbs light in the UV range, and a fluorophore.

When reagents and/or components comprising a kit are provided in a lyophilized form (lyophilisate) or as a dry powder, the lyophilisate or powder can be reconstituted by the addition of a suitable solvent. In particular aspects, the solvent may be a sterile, pharmaceutically acceptable buffer and/or other diluent. It is envisioned that such a solvent may also be provided as part of a kit.

When the components of a kit are provided in one and/or more liquid solutions, the liquid solution may be, by way of non-limiting example, a sterile, aqueous solution. The compositions may also be formulated into an administrative composition. In this case, the container means may itself be a syringe, pipette, topical applicator or the like, from which the formulation may be applied to an affected area of the body, injected into a subject, and/or applied to or mixed with the other components of the kit.

### XVIII. Examples

The following examples are given for the purpose of illustrating various aspects of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Hormad1 HLA-A1101 Restricted Peptide Identification, CTL Generation and TCR-T Development

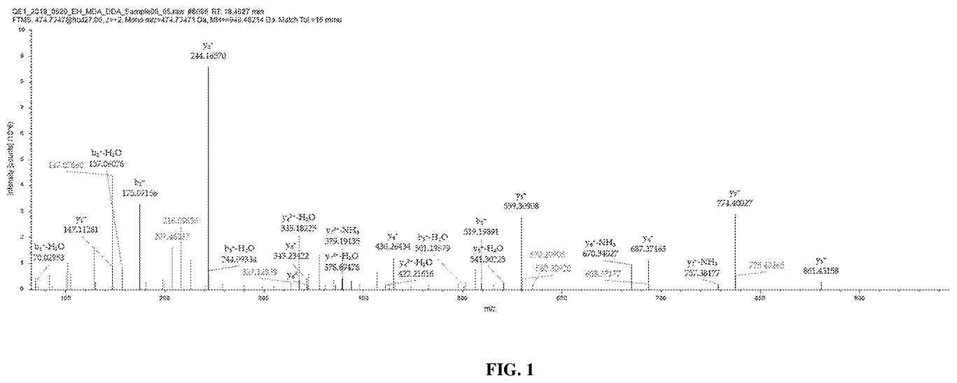

K562-A11-HORMAD1 (HLA-A1101 allele gene and HORMAD1 cDNA forced expression K562 cell line) cell line was lysed using NP-40 buffer and the lysate was incubated with Sepharose Fast Flow beads coupled with anti-MHC class I antibody. After washing to get rid of unbound protein, the MHC binding peptide was eluted with 10% acetic acid. The eluted peptide solution was concentrated and analyzed using tandem mass spectrometry (FIG. 1). One peptide (HMD-375, SSSQESVPK—SEQ ID NO:2) corresponded to the HORMAD1 sequence. The ion score is 60.

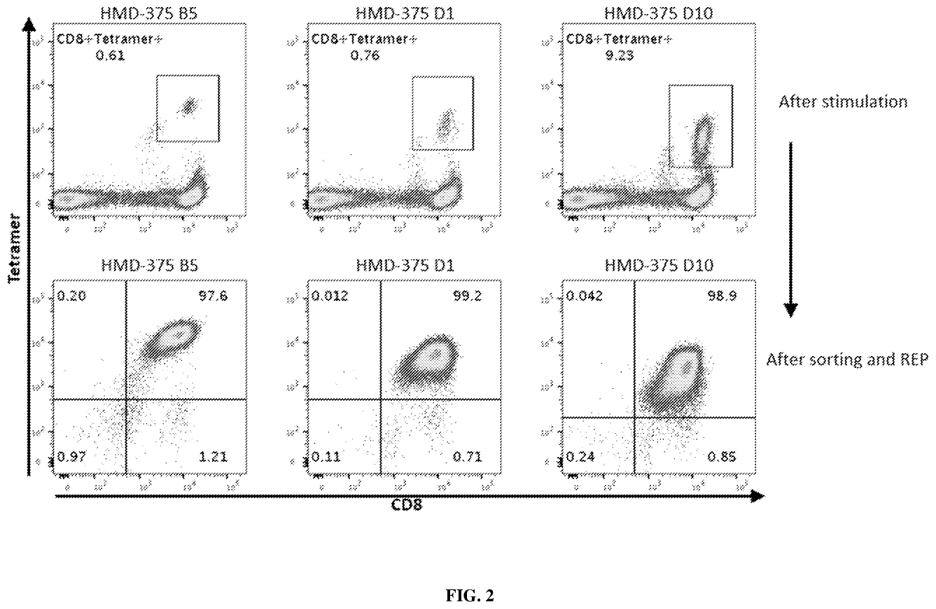

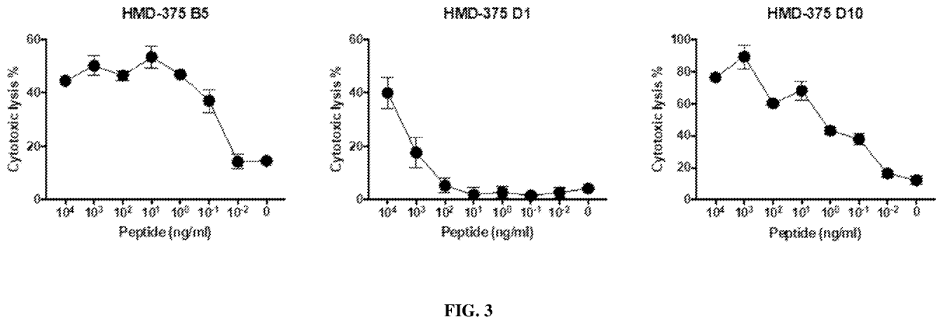

HMD-375 (SSSQESVPK—SEQ ID NO:2) peptide was pulsed to mature dendritic cells and then co-cultured with autologous PBMC from HLA-A1101 positive healthy donor in 48-well plates. After two rounds of stimulation, part of T cells from the each well were collected for flow cytometry detection with tetramer and anti-CD8 staining. A small CD8+/Tetramer+ population was observed in 3 wells. The T cells in the wells which showed tetramer+/CD8+ populations were picked up, and the tetramer+/CD8+ population were sorted and expanded with rapid expansion protocol (REP). After REP for two weeks, high purity cytotoxic T lymphocytes (CTLs—tetramer+ population over 90%) were observed (FIG. 2).

K562-A11 cells pulsed with various concentrations of HMD-375 peptide were used as targets. The lysis ability of different HMD-375 CTL cell lines was detected with Cr51 release assay (CRA) (FIG. 3). The effector to target (E:T) ratio is 20:1. HMD-375 B5 and HMD-375 D10 CTL cell lines showed good epitope recognition.

TABLE 2
Tumor cell line targets for HMD-375 CTL line killing assay

| | K562- | K562- | | H1299- | | | M14- | M14- |
|---|---|---|---|---|---|---|---|---|
| | eGFP | A11-HMD | H1299 | A11′ | H1623 | H647 | eGFP | HMD |
| Cancer | Leukemia | Leukemia | Lung | Lung | Lung | Lung | Melanoma | Melanoma |
| type | | | Ca | Ca | Ca | Ca | | |
| HLA-A1101 | + | + | − | + | + | + | + | + |
| HORMAD1 | − | + | + | + | + | − | − | + |

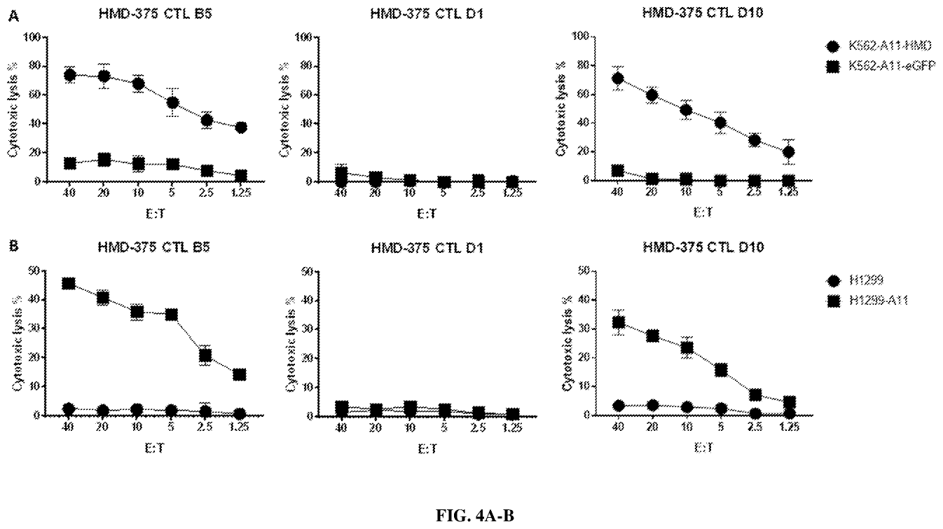

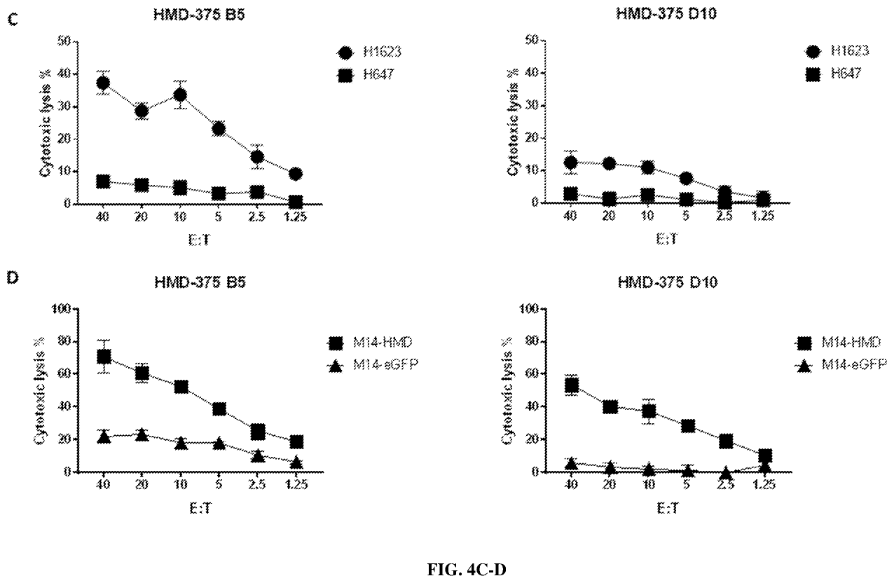

Different tumor cell lines were used as targets and co-cultured with different HMD-375 specific CTL cell lines (Table 2). The lysis ability of HMD-375 CTL cell lines was detected with Cr51 release assay (CRA). The effector to target (E:T) ratio was from 40:1 to 1.25:1. K562-A11 cell lines forced expressing eGFP or HORMAD1 were used as the targets (FIG. 4A). Tumor cell lines H1299 (HLA-A2−, HORMAD1+) and H1299-A11 (HLA-A11 forced expressing) (FIG. 4B); H1623 (HLA-A11+, HORMAD1+) and H647 (HLA-A11+, HORMAD1−) (FIG. 4C); and M14-eGFP (HLA-A11+, HORMAD1−, eGFP forced expressing) and M14-HMD (HLA-A11+, HORMAD1 forced expressing) (FIG. 4D) were used as targets. Both HMD-375 B5 CTL cell line and D10 CTL cell line show higher killing lever to positive targets compared with negative control (FIG. 4A-D).

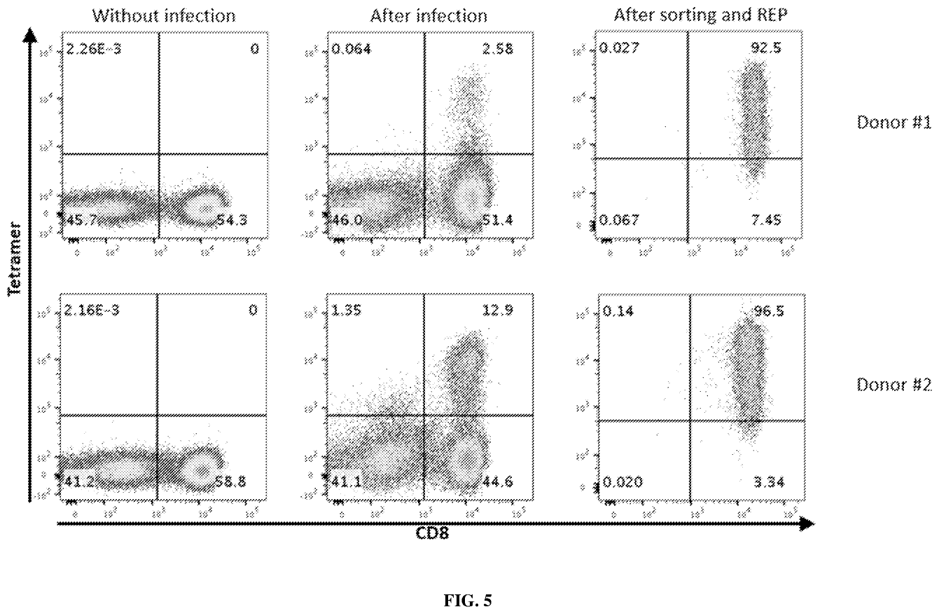

The whole length TCR alpha chain and beta chain were assembled with Furing-P2A skip peptide and inserted into retrovirus vector pMSGV3. The recombinant retrovirus vector were used to infect the healthy donor's PBMC. After infection, the CD8+/Tetramer+ population was appeared with the flow cytometry detection. After tetramer guided sorting and expansion, the high purity of TCR-T cell lines from two donors were generated (FIG. 5).

TABLE 3
TCR Cloning out and sequence analysis from HMD-375 B5 CTL cell line-Alpha
Chain

| | Productive TRA rearranged sequence | | |
|---|---|---|---|
| Results summary | (no stop codon and in-frame junction) | | |
| V-GENE and allele | Homsap TRAV38-2/ | Score = 1375 | Identity = 100.00% (276/276 nt) |
| | DV8*01 F | | |
| J-GENE and allele | Homsap TRAJ16*01 | Score = 234 | Identity = 92.59% (50/54 nt) |
| | F | | |
| FM-IMGT lengths, | [26.17.34.11] | [7.8.11] | CAYRSAVRQKLLF (SEQ ID |
| CDR-IMGT lengths | | | NO: 31) |
| and AA JUNCTION | | | |

TABLE 4
TCR Cloning out and sequence analysis from HMD-375 B5 CTL cell line-Beta Chain

| | Productive TRB rearranged sequence | | |
|---|---|---|---|
| Results summary | (no stop codon and in-frame junction) | | |
| V-GENE and allele | Homsap TRBV7- | Score = 1375 | Identity = 100.00% (276/276 nt) |
| | 9*03F | | |
| J-GENE and allele | Homsap TRBJ2-1*01 | Score = 241 | Identity = 98.00% (49/50 nt) |
| | F | | |
| D-GENE and allele by | Homsap TRBD2*01 | D-Region is in reading frame 2 | |
| IMGT/Junction | F | | |
| Analysis | | | |
| FM-IMGT lengths, | [26.17.38.10] | [5.6.14] | CASSSPGGRSYNEQFF (SEQ ID |
| CDR-IMGT lengths | | | NO: 32) |
| and AA JUNCTION | | | |

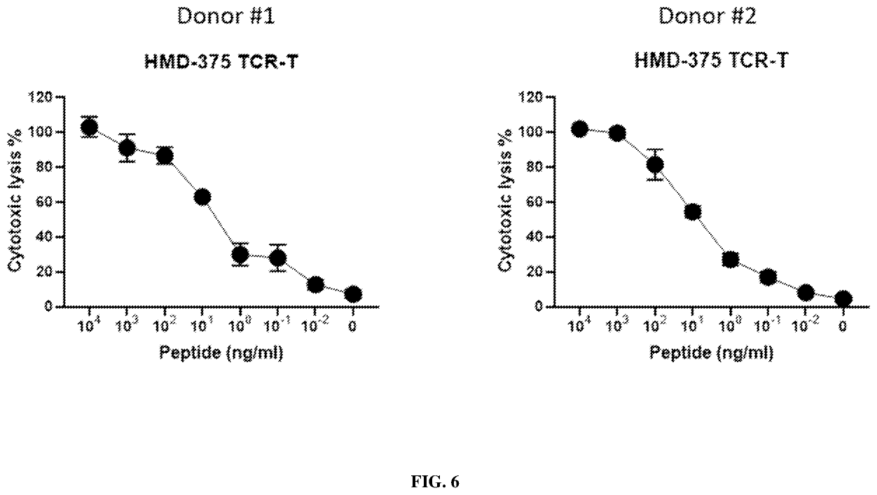

K562-A11 cells pulsed with various concentrations of HMD-375 peptide were used as targets. The lysis ability of different HMD-375 TCR-T was detected with Cr51 release assay (CRA) (FIG. 6). The effector to target (E:T) ratio is 20:1. Both HMD-375 TCR-T show good epitope's recognition.

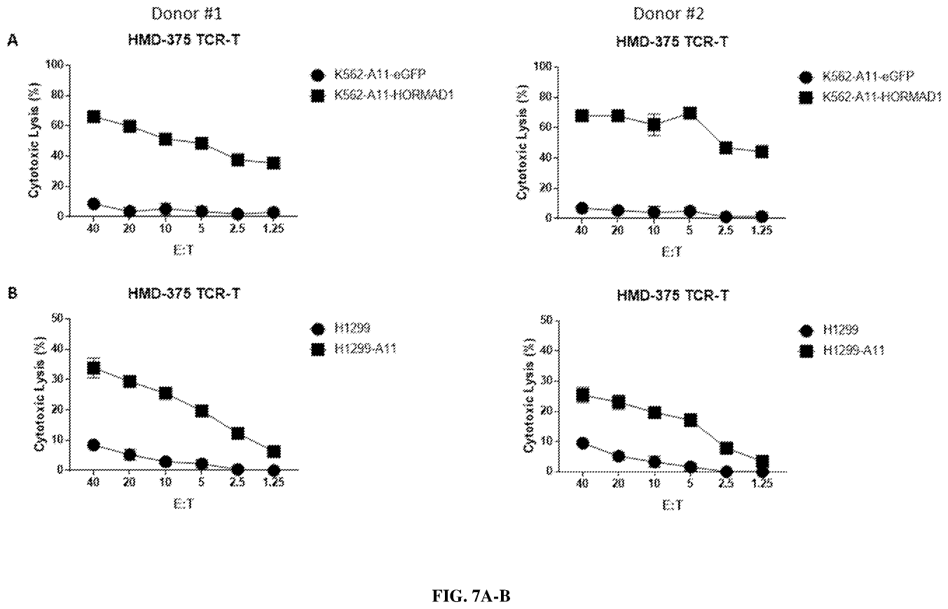

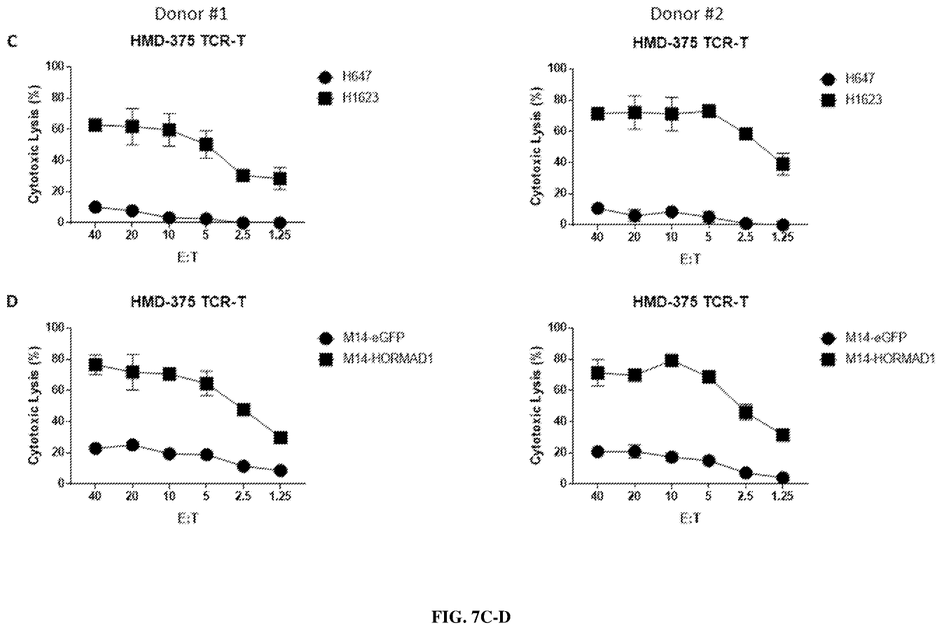

Different tumor cell lines were used as targets and co-cultured with different HMD-375 specific TCR-T cell lines. The lysis ability of HMD-375 TCR-T cell lines were detected with Cr51 release assay (CRA). The effector to target (E:T) ratio is from 40:1 to 1.25:1. K562-All cell lines forced expressing eGFP or HORMAD1 were as the targets (FIG. 7A). Tumor cell lines H1299 (HLA-A2−, HORMAD1+) and H1299-A11 (HLA-A11 forced expressing) (FIG. 7B); H1623 (HLA-A11+, HORMAD1+) and H647 (HLA-A11+, HORMAD1−) (FIG. 7C); and M14-eGFP (HLA-A11+, HORMAD1−, eGFP forced expressing) and M14-HMD (HLA-A11+, HORMAD1 forced expressing) (FIG. 7D) were used as targets. Both HMD-375 specific TCR-T cell lines from two healthy donors show higher killing lever to positive targets compared with negative control (FIG. 7A-D).

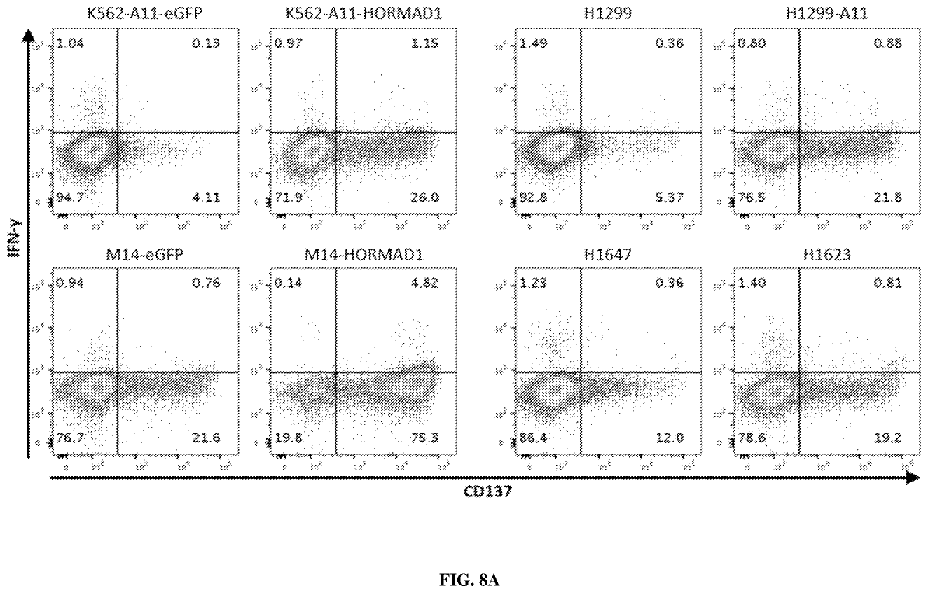

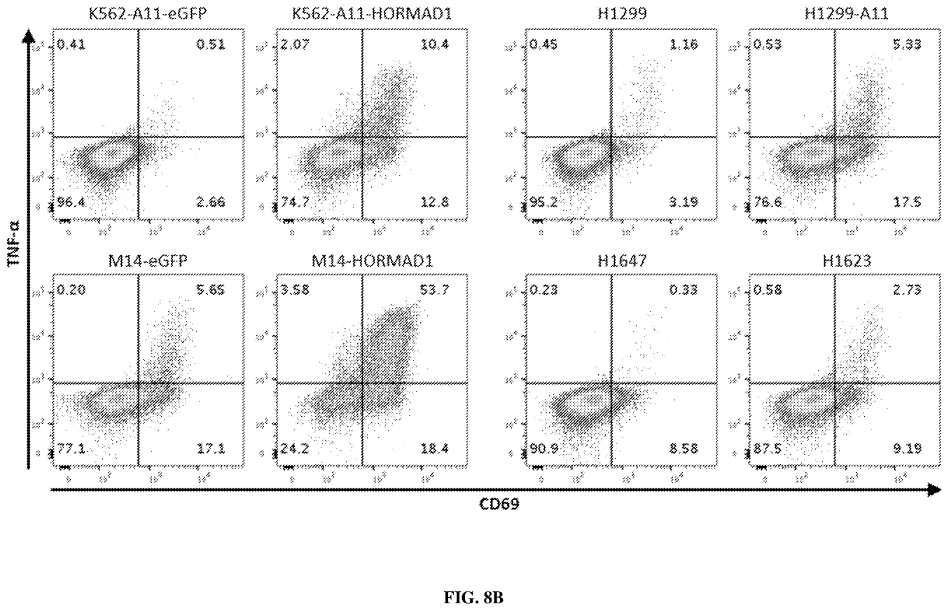

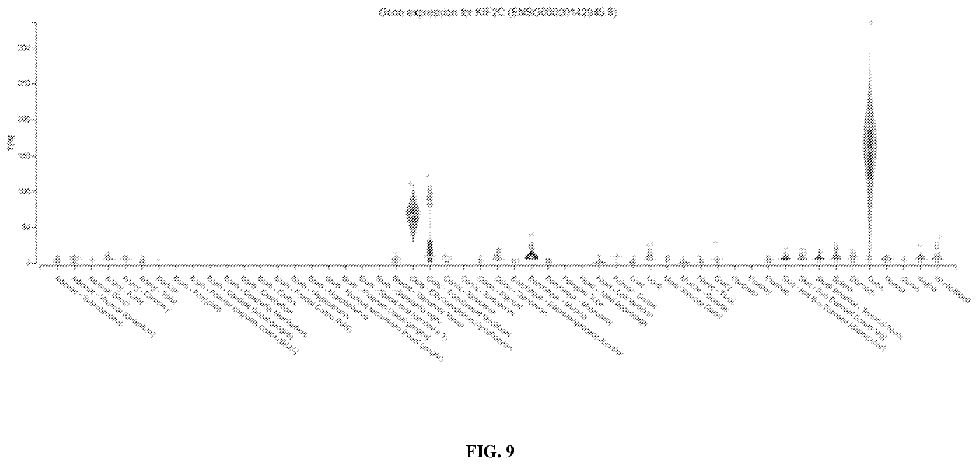

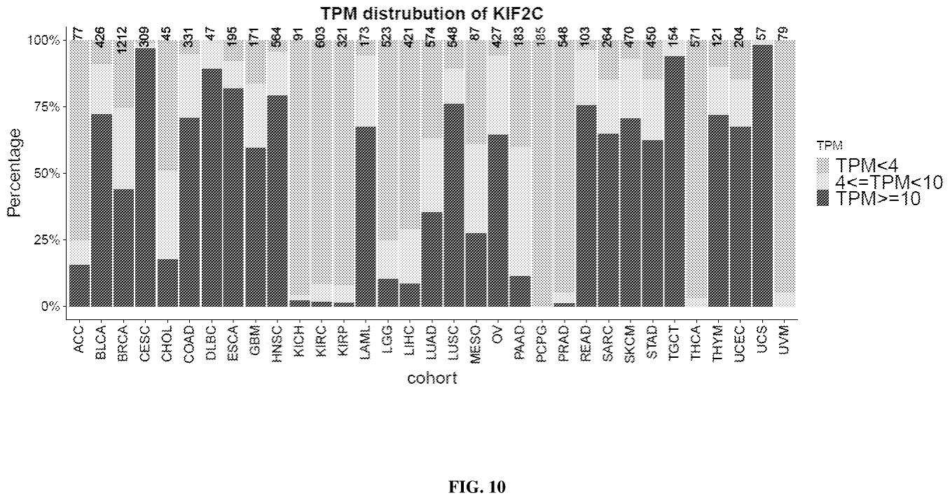

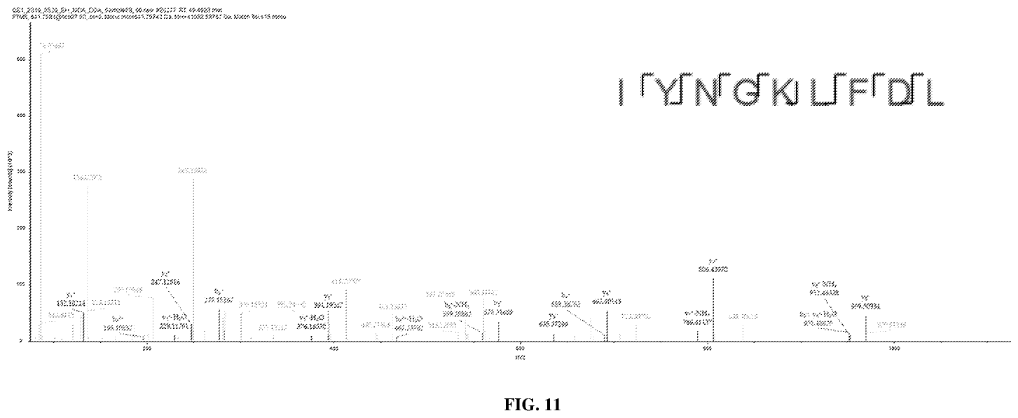

The HMD-375 specific TCR-T cell lines were co-cultured with K562-A11-eGFP, K562-A11-HORMAD1, H1299, H1299-A11, M14-eGFP, M14-HORMAD1, H647 and H1623 (E:T=10:1). After overnight co-culturing, the TCR pathway down-stream activated marker, CD137, CD69, IFN-γ and TNF-α were detected with ICS assay, the level of CD137, CD69, IFN-γ and TNF-α of HMD-375 specific TCR-T cell lines were significantly enhanced when they were co-cultured with positive targets K562-A11-HORMAD1, H1299-A11, M14-HORMAD1, and H1623, compared with negative targets (FIG. 8A-B).

### Example 2: KIF2C-404 (IYNGKLFDL—SEQ ID NO:1) Antigen Specific CTL Generation and TCR-T Development

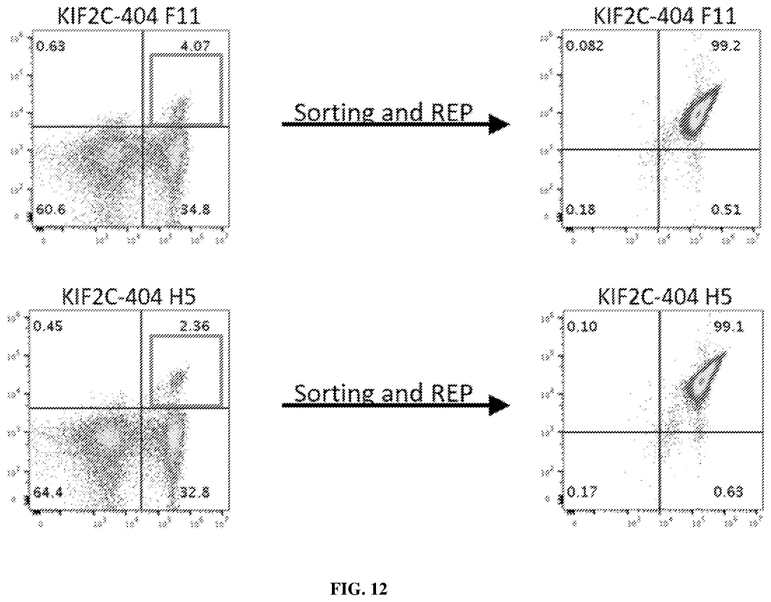

KIF2C-404 peptide (IYNGKLFDL—SEQ ID NO:1) was pulsed to mature dendritic cells and then co-cultured with autologous PBMC from HLA-A2402 positive healthy donor in the 48 well plates. After two rounds of stimulation, part of T cells from each well was collected for flow cytometry detection with tetramer and anti-CD8 staining. Small CD8+/Tetramer+ populations were observed in 2 wells. The T cells in the wells which show tetramer+/CD8+ populations were picked up, and the tetramer+/CD8+ population were sorted and expanded with rapid expansion protocol (REP). After REP for two weeks, high purity CTL (tetramer+ population over 90%) were observed (FIG. 12)

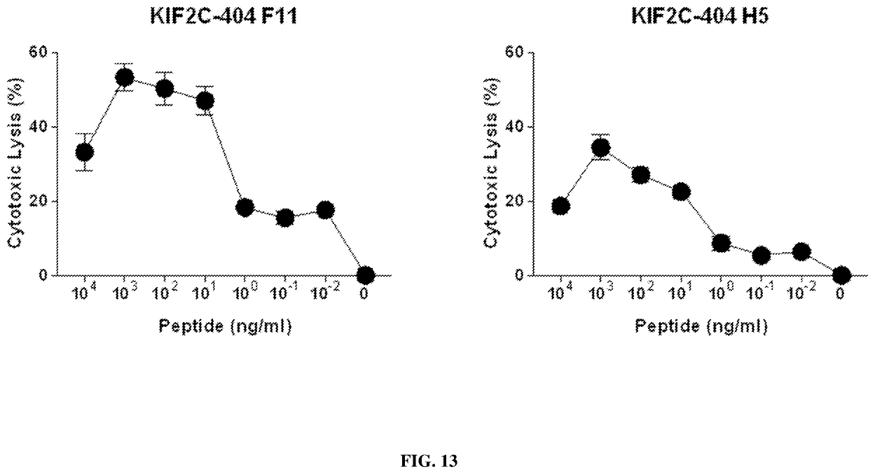

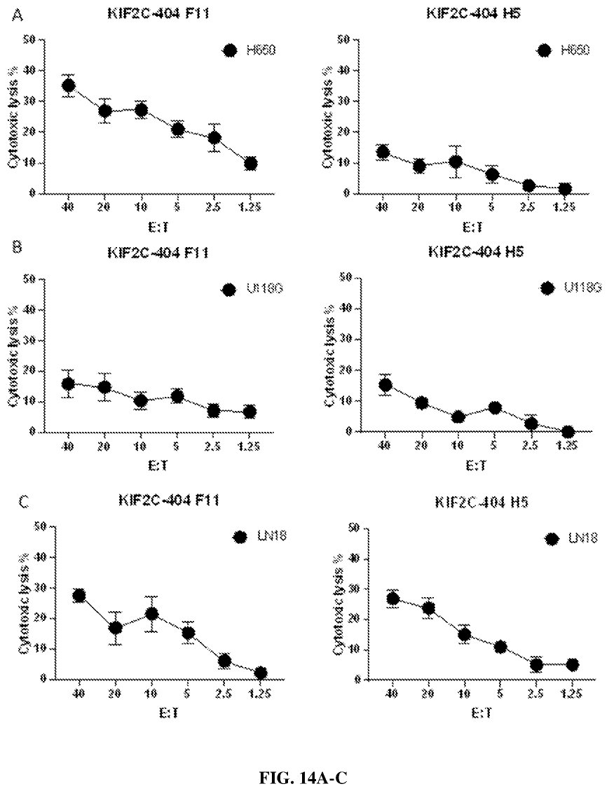

H1299 cells pulsed with various concentrations of KIF2C-404 peptide were used as targets. The lysis ability of different KIF2C-404 CTL cell lines was detected with 51Cr release assay (CRA (FIG. 13)). The effector to target (E:T) ratio is 20:1.

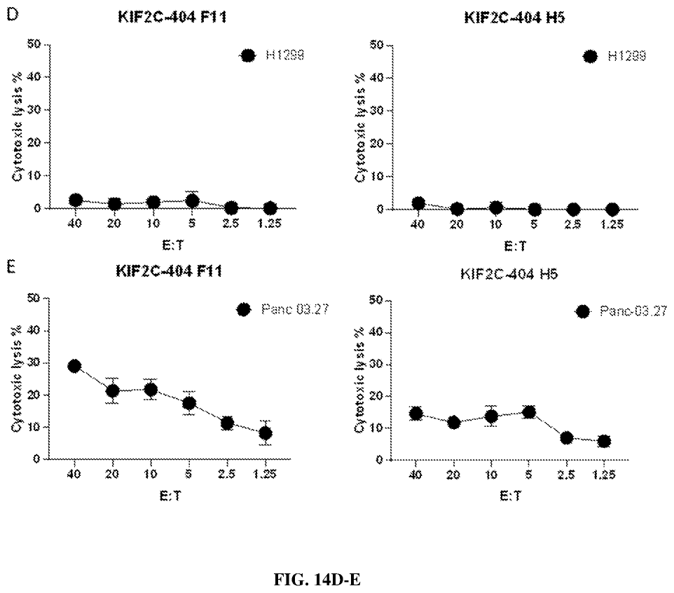

Different tumor cell lines were used as targets and co-cultured with KIF2C-404 specific CTL cell lines. The lysis ability of KIF2C-404 CTL cell lines were detected with CRA (FIG. 14). The effector to target (E:T) ratio is from 40:1 to 1.25:1.

TABLE 5
TCR Cloning out and sequence analysis from KIF2C-404 F11 CTL cell line-Alpha
Chain

| | Productive TRA rearranged sequence | | |
|---|---|---|---|
| Results summary | (no stop codon and in-frame junction) | | |
| V-GENE and allele | Homsap TRAV21*02 | Score = 1345 | Identity = 100.00% (270/270 nt) |
| | (F) | | |
| J-GENE and allele | Homsap TRAJ56*01 | Score = 292 | Identity = 96.77% (60/62 nt) |
| | F | | |
| FM-IMGT lengths, | [26.17.34.11] | [6.7.11] | CAVDTGANSKLTF (SEQ ID |
| CDR-IMGT lengths | | | NO: 33) |
| and AA JUNCTION | | | |

TABLE 6
TCR Cloning out and sequence analysis from KIF2C-404 F11 CTL cell line-Beta
Chain

| | Productive TRB rearranged sequence | | |
|---|---|---|---|
| Results summary | (no stop codon and in-frame junction) | | |
| V-GENE and allele | Homsap TRBV11- | Score = 1375 | Identity = 100.00% (276/276 nt) |
| | 2*03 F | | |
| J-GENE and allele | Homsap TRBJ1-2*01 | Score = 222 | Identity = 95.83% (46/48 nt) |
| | F | | |
| D-GENE and allele by | Homsap TRBD2*01 | D-Region is in reading frame 1 | |
| IMGT/Junction | F | | |
| Analysis | | | |
| FM-IMGT lengths, | [26.17.38.10] | [5.6.10] | CASSYHSYGYTF (SEQ ID |
| CDR-IMGT lengths | | | NO: 34) |
| and AA JUNCTION | | | |

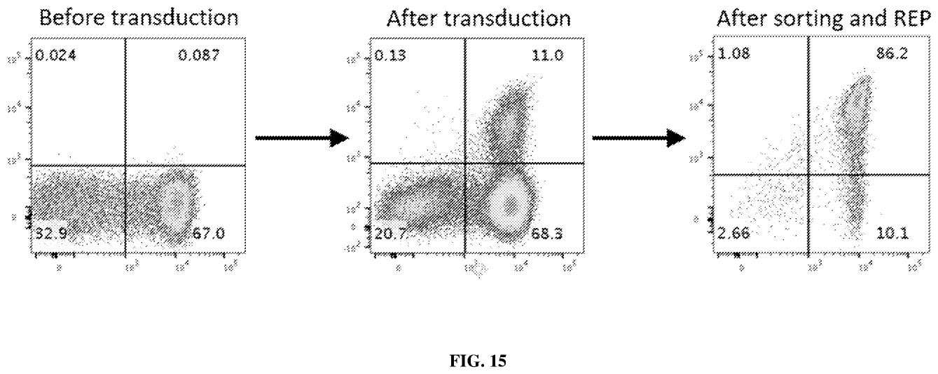

The whole length TCR alpha chain and beta chain were assembled with Furing-P2A skip peptide and inserted into retrovirus vector pMSGV3. The recombinant retrovirus vector were used to infect the healthy donor's PBMC. After infection, the CD8+/Tetramer+ population was appeared with the flow cytometry detection. After tetramer guided sorting and expansion, the high purity of TCR-T cell lines were generated (FIG. 15).

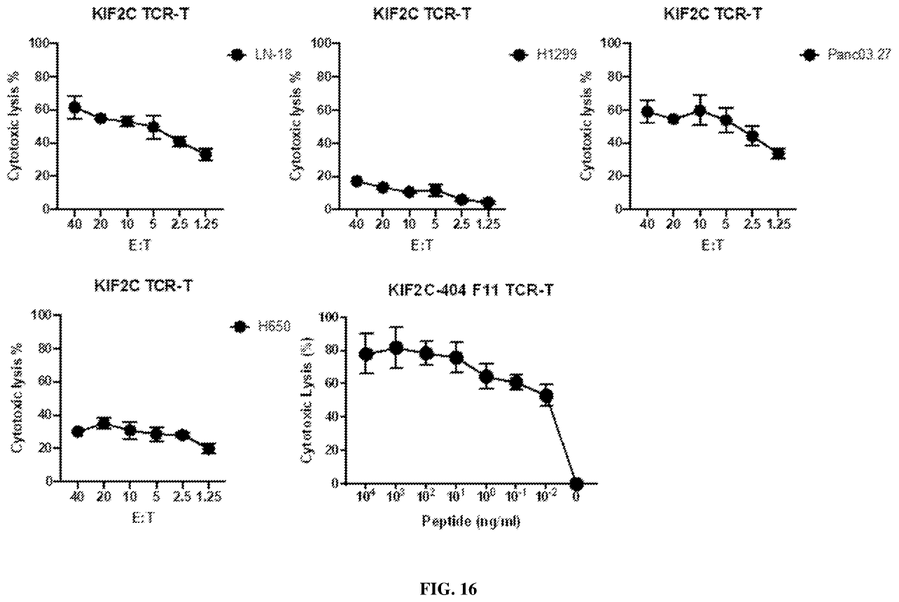

Different tumor cell lines, as well as peptide pulsed H1299 cells, were used as targets and co-cultured with KIF2C-404 specific TCR-T cell lines. The lysis ability of KIF2C-404 CTL cell lines were detected with CRA (FIG. 16).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A peptide comprising at least 70% sequence identity to a peptide of IYNGKLFDL (SEQ ID NO:1) or SSSQESVPK (SEQ ID NO:2).

2. The peptide of claim 1, wherein the peptide comprises SEQ ID NO:1 or 2.

3. The peptide of claim 1 or 2, wherein the peptide comprises at least 6 contiguous amino acids of SEQ ID NO:1 or 2.

4. The peptide of claim 1 or 3, wherein the peptide is 13 amino acids or fewer in length.

5. The peptide of claim 4, wherein the peptide consists of 9 amino acids.

6. The peptide of any one of claims 1-5, wherein the peptide is immunogenic.

7. The peptide of any one of claims 1-6, wherein the peptide is modified.

8. The peptide of claim 7, wherein the modification comprises conjugation to a molecule.

9. The peptide of claim 7 or 8, wherein the molecule comprises an antibody, a lipid, an adjuvant, or a detection moiety.

10. The peptide of any of claims 1-9, wherein the peptide has 1, 2 or 3 substitutions relative to a peptide of SEQ ID NO:1 or 2.

11. A molecular complex comprising the peptide of any one of claims 1-10 and a MHC polypeptide.

12. A pharmaceutical composition comprising the isolated peptide of any one of claims 1-10 or the molecular complex of claim 11 and a pharmaceutical carrier.

13. The pharmaceutical composition of claim 12, wherein the pharmaceutical composition is formulated for parenteral administration, intravenous injection, intramuscular injection, inhalation, or subcutaneous injection.

14. The pharmaceutical composition of claim 12 or 13, wherein the peptide is comprised in a liposome, lipid-containing nanoparticle, or in a lipid-based carrier.

15. The pharmaceutical composition of claim 14, wherein the pharmaceutical preparation is formulated for injection or inhalation as a nasal spray.

16. The pharmaceutical composition of any one of claims 12-15, wherein the composition is formulated as a vaccine.

17. The pharmaceutical composition of any one of claims 12-16, wherein the composition further comprises an adjuvant.

18. A nucleic acid encoding for the peptide of any one of claims 1-10.

19. An expression vector comprising the nucleic acid of claim 18.

20. A host cell comprising the nucleic acid of claim 18 or the expression vector of claim 19.

21. An in vitro isolated dendritic cell comprising the peptide of any one of claims 1-10, the nucleic acid of claim 18, or the expression vector of claim 19.

22. The dendritic cell of claim 21, wherein the dendritic cell is a mature dendritic cell.

23. The dendritic cell of claim 21 or 22, wherein the cell is a cell with an HLA-A type.

24. A peptide-specific binding molecule, wherein the molecule specifically binds to a peptide of any one of claim 1-10 or the molecular complex of claim 11.

25. The binding molecule of claim 24, wherein the molecule is bi-specific.

26. The binding molecule of claim 24 or 25, wherein the binding molecule is an antibody, TCR mimc antibody, scFV, camelid, aptamer, or DARPIN.

27. A method of making a cell comprising transferring the nucleic acid of claim 18 or the expression vector of claim 19 into the cell.

28. The method of claim 27, wherein the method further comprises isolating the expressed peptide or polypeptide.

29. A method of producing cancer-specific immune effector cells comprising:
(a) obtaining a starting population of immune effector cells; and
(b) contacting the starting population of immune effector cells with a peptide of any one of claims 1-10 or the molecular complex of claim 11, thereby generating peptide-specific immune effector cells.

30. The method of claim 29, wherein contacting is further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface.

31. The method of claim 30, wherein the APCs are dendritic cells.

32. The method of any one of claims 29-31, wherein the immune effector cells are T cells, peripheral blood lymphocytes, NK cells, invariant NK cells, NKT cells.

33. The method of any one of claims 29-32, wherein the immune effector cells have been differentiated from mesenchymal stem cell (MSC) or induced pluripotent stem (iPS) cells.

34. The method of claim 32, wherein the T cells are CD8⁺ T cells, CD4⁺ T cells, CD4⁺ Treg cells, or γδ T cells.

35. The method of claim 32, wherein the T cells are cytotoxic T lymphocytes (CTLs).

36. The method of any one of claims 29-35, wherein obtaining comprises isolating the starting population of immune effector cells from peripheral blood mononuclear cells (PBMCs).

37. The method of any one of claims 29-36, wherein the starting population of immune effector cells is obtained from a subject.

38. The method of claim 37, wherein the subject is a human.

39. The method of claim 37 or 38, wherein the subject has a cancer.

40. The method of claim 39, wherein the cancer comprises tumor cells that are positive for expression of the peptide.

41. The method of any one of claims 1-40, wherein the cancer comprises leukemia, lung cancer, or skin cancer.

42. The method of any one of claims 29-41, wherein the cancer comprises a solid tumor.

43. The method of any one of claims 29-42, wherein the cancer comprises lung cancer.

44. The method of claim 43, wherein the lung cancer comprises adenocarcinoma or squamous cell carcinoma.

45. The method of any one of claims 31-41, wherein the method further comprises introducing the peptide or a nucleic acid encoding the peptide into the dendritic cells prior to the co-culturing.

46. The method of claim 45, where the peptide or nucleic acids encoding the peptide are introduced by electroporation.

47. The method of claim 45, wherein the peptide or nucleic acids encoding the peptide are introduced by adding the peptide or nucleic acid encoding the peptide to the dendritic cell culture media.

48. The method of claim 45, wherein the immune effector cells are co-cultured with a second population of dendritic cells into which the peptide or the nucleic acid encoding the peptide has been introduced.

49. The method of claim 45, wherein a population of CD8 or CD4-positive and peptide MHC tetramer-positive T cells are purified from the immune effector cells following the co-culturing.

50. The method of claim 49, wherein a clonal population of peptide-specific immune effector cells are generated by limiting or serial dilution followed by expansion of individual clones by a rapid expansion protocol.

51. The method of claim 50, wherein the method further comprises cloning of a T cell receptor (TCR) from the clonal population of peptide-specific immune effector cells.

52. The method of claim 51, wherein cloning of the TCR is cloning of a TCR alpha and a beta chain.

53. The method of claim 51 or claim 52, wherein the TCR is cloned using a 5′-Rapid amplification of cDNA ends (RACE) method.

54. The method of claim 53, wherein the cloned TCR is subcloned into an expression vector.

55. The method of claim 54, wherein the expression vector is a retroviral or lentiviral vector.

56. The method of claim 55, where a host cell is transduced with the expression vector to generate an engineered cell that expresses the TCR.

57. The method of claim 56, wherein the host cell is an immune cell.

58. The method of any one of claims 31-57, wherein the immune cell is a T cell and the engineered cell is an engineered T cell.

59. The method of claim 58, wherein the T cell is a CD8⁺ T cell, CD4+ T cell, CD4⁺ Treg cells, or γδ T cell and the engineered cell is an engineered T cell.

60. The method of claim 59, wherein the starting population of immune effector cells is obtained from a subject with cancer and the host cell is allogeneic or autologous to the subject.

61. The method of claim 60, wherein the cancer is positive for expression of the peptide.

62. The method of claim 58 or 59, wherein a population of CD8 or CD4-positive and peptide MHC tetramer-positive engineered T cells are purified from the transduced host cells.

63. The method of claim 49, wherein a clonal population of peptide-specific engineered T cells are generated by limiting or serial dilution followed by expansion of individual clones by a rapid expansion protocol.

64. A peptide-specific engineered T cell produced according to any one of the methods of claims 29-63.

65. A polypeptide comprising an antigen binding variable region comprising a CDR3 comprising the amino acid sequence of SEQ ID NO:9 or an amino acid sequence with at least 80% sequence identity to SEQ ID NO:9.

66. The polypeptide of claim 65, wherein the variable region comprises a CDR1, CDR2, and/or CDR3.

67. The polypeptide of claim 66, wherein the variable region comprises a CDR1 and/or CDR2 with the amino acid sequence of SEQ ID NO:7 and SEQ ID NO:8, respectively, or with at least 80% sequence identity to SEQ ID NO:7 and SEQ ID NO:8, respectively.

68. The polypeptide of any one of claims 65-67, wherein the variable region comprises the amino acid sequence of SEQ ID NO:5 or an amino acid sequence with at least 70% sequence identity to SEQ ID NO:5.

69. The polypeptide of any one of claims 65-68, wherein the polypeptide comprises a T cell receptor alpha (TCR-a) variable region.

70. The polypeptide of claim 69, wherein the polypeptide comprises a TCR-a variable and constant region.

71. The polypeptide of any one of claims 65-70, wherein the polypeptide further comprises a signal peptide.

72. The polypeptide of claim 71, wherein the signal peptide comprises the amino acid sequence of SEQ ID NO:6 or an amino acid sequence with at least 80% identity to SEQ ID NO:6.

73. A polypeptide comprising an antigen binding variable region comprising a CDR3 comprising the amino acid sequence of SEQ ID NO:16 or an amino acid sequence with at least 80% sequence identity to SEQ ID NO:16.

74. The polypeptide of claim 73, wherein the variable region comprises a CDR1, CDR2, and/or CDR3.

75. The polypeptide of claim 74, wherein the variable region comprises a CDR1 and/or CDR2 with the amino acid sequence of SEQ ID NO: 14 and 15, respectively, or with an amino acid sequence that is at least 80% sequence identity to SEQ ID NO:14 and SEQ ID NO:15, respectively.

76. The polypeptide of any one of claims 73-75, wherein the variable region comprises an amino acid sequence or SEQ ID NO:12 or an amino acid sequence with at least 70% sequence identity to SEQ ID NO:12.

77. The polypeptide of any one of claims 73-76, wherein the polypeptide comprises a T cell receptor beta (TCR-b) variable region.

78. The polypeptide of claim 77, wherein the polypeptide comprises a TCR-b variable and constant region.

79. The polypeptide of any one of claims 73-78, wherein the polypeptide further comprises a signal peptide.

80. The polypeptide of claim 79, wherein the signal peptide comprises the amino acid sequence of SEQ ID NO:13 or an amino acid sequence with at least 80% identity to SEQ ID NO:13.

81. The polypeptide of any one of claims 65-80, wherein the polypeptide comprises a TCR-a and TCR-b variable region.

82. The polypeptide of any one of claims 65-81, wherein the polypeptide is recombinant.

83. A polypeptide comprising an antigen binding variable region comprising a CDR3 comprising the amino acid sequence of SEQ ID NO:23 or an amino acid sequence with at least 80% sequence identity to SEQ ID NO:23.

84. The polypeptide of claim 83, wherein the variable region comprises a CDR1, CDR2, and/or CDR3.

85. The polypeptide of claim 84, wherein the variable region comprises a CDR1 and/or CDR2 with the amino acid sequence of SEQ ID NO:21 and SEQ ID NO:22, respectively, or with at least 80% sequence identity to SEQ ID NO:21 and SEQ ID NO:22, respectively.

86. The polypeptide of any one of claims 83-85, wherein the variable region comprises the amino acid sequence of SEQ ID NO:19 or an amino acid sequence with at least 70% sequence identity to SEQ ID NO:19.

87. The polypeptide of any one of claims 83-86, wherein the polypeptide comprises a T cell receptor alpha (TCR-a) variable region.

88. The polypeptide of claim 87, wherein the polypeptide comprises a TCR-a variable and constant region.

89. The polypeptide of any one of claims 83-88, wherein the polypeptide further comprises a signal peptide.

90. The polypeptide of claim 89, wherein the signal peptide comprises the amino acid sequence of SEQ ID NO:20 or an amino acid sequence with at least 80% identity to SEQ ID NO:20.

91. A polypeptide comprising an antigen binding variable region comprising a CDR3 comprising the amino acid sequence of SEQ ID NO:30 or an amino acid sequence with at least 80% sequence identity to SEQ ID NO:30.

92. The polypeptide of claim 91, wherein the variable region comprises a CDR1, CDR2, and/or CDR3.

93. The polypeptide of claim 92, wherein the variable region comprises a CDR1 and/or CDR2 with the amino acid sequence of SEQ ID NO:28 and 29, respectively, or with an amino acid sequence that is at least 80% sequence identity to SEQ ID NO:28 and SEQ ID NO:29, respectively.

94. The polypeptide of any one of claims 91-93, wherein the variable region comprises an amino acid sequence or SEQ ID NO:26 or an amino acid sequence with at least 70% sequence identity to SEQ ID NO:26.

95. The polypeptide of any one of claims 91-94, wherein the polypeptide comprises a T cell receptor beta (TCR-b) variable region.

96. The polypeptide of claim 95, wherein the polypeptide comprises a TCR-b variable and constant region.

97. The polypeptide of any one of claims 91-96, wherein the polypeptide further comprises a signal peptide.

98. The polypeptide of any one of claims 83-97, wherein the polypeptide comprises a TCR-a and TCR-b variable region.

99. The polypeptide of any one of claims 83-98, wherein the polypeptide is recombinant.

100. The polypeptide of claim 97, wherein the signal peptide comprises the amino acid sequence of SEQ ID NO:27 or an amino acid sequence with at least 80% identity to SEQ ID NO:27.

101. A nucleic acid encoding for the polypeptide of any one of claims 65-100.

102. The nucleic acid of claim 101, wherein the nucleic acid is a cDNA.

103. An engineered T-cell Receptor (TCR) comprising a TCR-a region and a TCR-b region, wherein the TCR-a region comprises a CDR3 with the amino acid sequence of SEQ ID NO:9 or an amino acid sequence with at least 80% sequence identity to SEQ ID NO:9 and the TCR-b region comprises a CDR3 with the amino acid sequence of SEQ ID NO: 16 or an amino acid sequence with at least 80% sequence identity to SEQ ID NO:16.

104. The TCR of claim 103, wherein the TCR comprises a TCR-a region comprising a variable region comprising CDR1, CDR2, and CDR3 and a TCR-b region comprising a variable region comprising CDR1, CDR2, and CDR3.

105. The TCR of claim 104, wherein the TCR-a region comprises a CDR1 having the amino acid sequence of SEQ ID NO:7 or having an amino acid sequence with at least 80% sequence identity to SEQ ID NO:7 and/or the TCR-b region comprises a CDR1 having the amino acid sequence of SEQ ID NO:14 or having an amino acid sequence with at least 80% sequence identity to SEQ ID NO:14.

106. The TCR of claim 104 or 105, wherein the TCR-a region comprises a CDR2 having the amino acid sequence of SEQ ID NO:8 or having an amino acid sequence with at least 80% sequence identity to SEQ ID NO:8 and/or the TCR-b region comprises a CDR2 having the amino acid sequence of SEQ ID NO:15 or having an amino acid sequence with at least 80% sequence identity to SEQ ID NO:15.

107. The TCR of any one of claims 103-106, wherein the CDR1, CDR2, and CDR3 of the TCR-a region comprise the amino acid sequence of SEQ ID NO:7, 8, and 9, respectively and wherein the CDR1, CDR3, and CDR3 of the TCR-b region comprise the amino acid sequence of SEQ ID NO:14, 15, and 16, respectively.

108. The TCR of any one of claims 103-107, wherein the TCR-a region comprises the amino acid sequence of SEQ ID NO:5 or an amino acid sequence with at least 70% sequence identity to SEQ ID NO:5 and the TCR-b region comprises the amino acid sequence of SEQ ID NO:12 or an amino acid sequence with at least 70% sequence identity to SEQ ID NO:12.

109. An engineered T-cell Receptor (TCR) comprising a TCR-a region and a TCR-b region, wherein the TCR-a region comprises a CDR3 with the amino acid sequence of SEQ ID NO:23 or an amino acid sequence with at least 80% sequence identity to SEQ ID NO:23 and the TCR-b region comprises a CDR3 with the amino acid sequence of SEQ ID NO:30 or an amino acid sequence with at least 80% sequence identity to SEQ ID NO:30.

110. The TCR of claim 109, wherein the TCR comprises a TCR-a region comprising a variable region comprising CDR1, CDR2, and CDR3 and a TCR-b region comprising a variable region comprising CDR1, CDR2, and CDR3.

111. The TCR of claim 110, wherein the TCR-a region comprises a CDR1 having the amino acid sequence of SEQ ID NO:21 or having an amino acid sequence with at least 80% sequence identity to SEQ ID NO:21 and/or the TCR-b region comprises a CDR1 having the amino acid sequence of SEQ ID NO:28 or having an amino acid sequence with at least 80% sequence identity to SEQ ID NO:28.

112. The TCR of claim 110 or 111, wherein the TCR-a region comprises a CDR2 having the amino acid sequence of SEQ ID NO:22 or having an amino acid sequence with at least 80% sequence identity to SEQ ID NO:22 and/or the TCR-b region comprises a CDR2 having the amino acid sequence of SEQ ID NO:29 or having an amino acid sequence with at least 80% sequence identity to SEQ ID NO:29.

113. The TCR of any one of claims 109-112, wherein the CDR1, CDR2, and CDR3 of the TCR-a region comprise the amino acid sequence of SEQ ID NO:21, 22, and 23, respectively and wherein the CDR1, CDR3, and CDR3 of the TCR-b region comprise the amino acid sequence of SEQ ID NO:28, 29, and 30, respectively.

114. The TCR of any one of claims 109-113, wherein the TCR-a region comprises the amino acid sequence of SEQ ID NO: 19 or an amino acid sequence with at least 70% sequence identity to SEQ ID NO: 19 and the TCR-b region comprises the amino acid sequence of SEQ ID NO:26 or an amino acid sequence with at least 70% sequence identity to SEQ ID NO:26.

115. The TCR of any one of claims 103-114, wherein the TCR is a single chain TCR.

116. The TCR of any one of claims 103-114, wherein the TCR-a region and the TCR-b region are on different polypeptides.

117. The TCR of any one of claims 103-114, wherein the TCR-a region and the TCR-b region are on the same polypeptide.

118. A polypeptide comprising the TCR of any one of claims 103-117, wherein the polypeptide is bi-specific.

119. A fusion protein comprising the TCR of any one of claims 103-118 and a CD3 binding region.

120. The fusion protein of claim 119, wherein the CD3 binding region comprises a CD3-specific fragment antigen binding (Fab), single chain variable fragment (scFv), single domain antibody, or single chain antibody.

121. A nucleic acid encoding a TCR-a polypeptide and/or a TCR-b polypeptide, wherein the TCR-a polypeptide comprises a CDR3 with the amino acid sequence of SEQ ID NO:9 or an amino acid sequence with at least 80% sequence identity to SEQ ID NO:9 and the TCR-b polypeptide comprises a CDR3 with the amino acid sequence of SEQ ID NO:16 or an amino acid sequence with at least 80% sequence identity to SEQ ID NO:16.

122. The nucleic acid of claim 121, wherein the nucleic acid encodes for a TCR-a polypeptide comprising a CDR1, CDR2, and CDR3 and/or a TCR-b polypeptide comprising a CDR1, CDR2, and CDR3.

123. The nucleic acid of claim 122, wherein the nucleic acid encodes for a TCR-a polypeptide comprising a CDR1 having the amino acid sequence of SEQ ID NO:7 or having an amino acid sequence with at least 80% sequence identity to SEQ ID NO:7 and/or a TCR-b polypeptide comprising a CDR1 having the amino acid sequence of SEQ ID NO: 14 or having an amino acid sequence with at least 80% sequence identity to SEQ ID NO:14.

124. The nucleic acid of claim 122 or 123, wherein the nucleic acid encodes for TCR-a polypeptide comprising a CDR2 having the amino acid sequence of SEQ ID NO:8 or having an amino acid sequence with at least 80% sequence identity to SEQ ID NO:8 and/or a TCR-b polypeptide comprising a CDR2 having the amino acid sequence of SEQ ID NO: 15 or having an amino acid sequence with at least 80% sequence identity to SEQ ID NO:15.

125. The nucleic acid of any one of claims 109-124, wherein the nucleic acid encodes a TCR-a variable region having the amino acid sequence of SEQ ID NO:5 or an amino acid sequence with at least 70% sequence identity to SEQ ID NO:5 and/or a TCR-b variable region having the amino acid sequence of SEQ ID NO: 12 or an amino acid sequence with at least 70% sequence identity to SEQ ID NO:12.

126. The nucleic acid of claim 125, wherein the nucleic acid encodes a TCR-a polypeptide having the amino acid sequence of SEQ ID NO:4 or an amino acid sequence with at least 70% sequence identity to SEQ ID NO:4 and/or a TCR-b polypeptide chain having the amino acid sequence of SEQ ID NO:11 or an amino acid sequence with at least 70% sequence identity to SEQ ID NO:11.

127. The nucleic acid of claim 126, wherein the nucleic acid comprises SEQ ID NO:3, 10, and/or a fragment thereof.

128. A nucleic acid encoding a TCR-a polypeptide comprising a CDR3 with the amino acid sequence of SEQ ID NO:23 or an amino acid sequence with at least 80% sequence identity to SEQ ID NO:23 and/or a TCR-b polypeptide comprising a CDR3 with the amino acid sequence of SEQ ID NO:30 or an amino acid sequence with at least 80% sequence identity to SEQ ID NO:30.

129. The nucleic acid of claim 128, wherein the nucleic acid encodes for a TCR-a polypeptide comprising a CDR1, CDR2, and CDR3 and/or a TCR-b polypeptide comprising a CDR1, CDR2, and CDR3.

130. The nucleic acid of claim 129, wherein the nucleic acid encodes for a TCR-a comprising a CDR1 having the amino acid sequence of SEQ ID NO:21 or having an amino acid sequence with at least 80% sequence identity to SEQ ID NO:21 and/or a TCR-b polypeptide comprising a CDR1 having the amino acid sequence of SEQ ID NO:28 or having an amino acid sequence with at least 80% sequence identity to SEQ ID NO:28.

131. The nucleic acid of claim 129 or 130, wherein the nucleic acid encodes for a TCR-a polypeptide comprising a CDR2 having the amino acid sequence of SEQ ID NO:22 or having an amino acid sequence with at least 80% sequence identity to SEQ ID NO:22 and/or a TCR-b polypeptide comprising a CDR2 having the amino acid sequence of SEQ ID NO:29 or having an amino acid sequence with at least 80% sequence identity to SEQ ID NO:29.

132. The nucleic acid of any one of claims 128-131, wherein the nucleic acid encodes a TCR-a variable region having the amino acid sequence of SEQ ID NO:19 or an amino acid sequence with at least 70% sequence identity to SEQ ID NO:19 and/or a TCR-b variable region having the amino acid sequence of SEQ ID NO:26 or an amino acid sequence with at least 70% sequence identity to SEQ ID NO:26.

133. The nucleic acid of claim 132, wherein the nucleic acid encodes a TCR-a polypeptide chain having the amino acid sequence of SEQ ID NO:18 or an amino acid sequence with at least 70% sequence identity to SEQ ID NO:18 and/or a TCR-b polypeptide chain having the amino acid sequence of SEQ ID NO:25 or an amino acid sequence with at least 70% sequence identity to SEQ ID NO:25.

134. The nucleic acid of claim 133, wherein the nucleic acid comprises SEQ ID NO:17, 24, and/or a fragment thereof.

135. The nucleic acid of any one of claims 121-134, wherein the nucleic acid comprises at least one TCR-a (TRA) and at least one TCR-b (TRB) gene on the same nucleic acid.

136. The nucleic acid of any one of claims 121-135, wherein the nucleic acid is polycistronic.

137. The nucleic acid of claim 136, wherein the nucleic acid comprises an internal ribosome entry site (IRES) or a 2A cleavable linker.

138. The nucleic acid(s) of any one of claims 121-137, wherein the nucleic acid comprises a cDNA encoding the TCR-a and/or TCR-b genes.

139. The nucleic acid(s) of any one of claims 121-138, wherein the nucleic acid further encodes for a polypeptide comprising a CD3 binding region.

140. The nucleic acid of claim 139, wherein the CD3 binding region comprises a CD3-specific fragment antigen binding (Fab), single chain variable fragment (scFv), single domain antibody, or single chain antibody.

141. The nucleic acid of any one of claims 121-140, wherein the nucleic acid is a cDNA.

142. A nucleic acid vector comprising the nucleic acid(s) of any one of claim 101, 102, or 121-141.

143. The vector of claim 142, wherein the vector comprises a promoter that directs the expression of the nucleic acid.

144. The vector of claim 143, wherein the promoter comprises a murine stem cell virus (MSCV) promoter.

145. The vector of any one of claims 142-144, wherein the vector comprises the TCR-a and TCR-b genes.

146. A cell comprising the polypeptide of any one of claims 65-100, TCR of any one of claims 103-114, the fusion protein of claim 115 or 118, the nucleic acid(s) of any one of claim 101, 102, or 121-141, or the vector of any one of claims 142-145.

147. The cell of claim 146, wherein the cell comprises a stem cell, a progenitor cell, an immune cell, or a natural killer (NK) cell.

148. The cell of claim 147, wherein the cell comprises a hematopoietic stem or progenitor cell, a T cell, a cell differentiated from mesenchymal stem cells (MSCs) or an induced pluripotent stem cell (iPSC).

149. The cell of claim 146 or 147, wherein the cell is isolated or derived from peripheral blood mononuclear cell (PBMCs).

150. The cell of claim 148 or 149, wherein the T cell comprises a cytotoxic T lymphocyte (CTL), a CD8⁺ T cell, a CD4⁺ T cell, an invariant NK T (INKT) cell, a gamma-delta T cell, a NKT cell, a regulatory T cell, or a CD4⁺ regulatory cell.

151. The cell of any one of claims 146-150, wherein the cell is isolated from a cancer patient.

152. A composition comprising the peptide-specific T cells of claim 64, the polypeptide of any one of claim 65-100 or 118, the TCR of any one of claims 103-115, the fusion protein of claim 119 or 120, the nucleic acid(s) of any one of claim 101, 102, or 121-141, or the vector of any one of claims 142-145.

153. The composition of claim 152, wherein the composition has been determined to be serum-free, mycoplasma-free, endotoxin-free, and/or sterile.

154. A method of making an engineered cell comprising transferring the nucleic acid of any one of claim 101, 102, or 121-141 or the vector of any one of claims 142-145 into a cell.

155. The method of claim 154, wherein the method further comprises culturing the cell in media, incubating the cell at conditions that allow for the division of the cell, screening the cell, and/or freezing the cell.

156. A method of treating or preventing cancer in a subject, the method comprising administering an effective amount of the peptide of any one of claims 1-10 or the molecular complex of claim 11, the pharmaceutical composition of any one of claim 12-17 or 152-155, the nucleic acid or expression vector of any one of claim 18, 19, or 142-145, the dendritic cell of any one of claims 21-23, or the peptide-specific T cells of claim 64, the polypeptide of any one of claim 65-100 or 118, the TCR of any one of claims 103-115, the fusion protein of claim 119 or 120 to the subject.

157. A method of stimulating an immune response in a subject, the method comprising administering an effective amount of the peptide of any one of claims 1-10 or the molecular complex of claim 11, the pharmaceutical composition of any one of claim 12-17 or 152-155, the nucleic acid or expression vector of any one of claim 18, 19, or 142-145, the dendritic cell of any one of claims 21-23, or the peptide-specific T cells of claim 64, the polypeptide of any one of claim 65-100 or 118, the TCR of any one of claims 103-115, the fusion protein of claim 119 or 120 to the subject.

158. The method of claim 156 or 157, wherein the subject is a human.

159. The method of any one of claims 156-158, wherein the peptide-specific T cells are autologous or allogeneic.

160. The method of any one of claims 156-159, further comprising administering at least a second therapeutic agent.

161. The method of claim 160, wherein the second therapeutic agent is an anti-cancer agent.

162. The method of any one of claims 156-161, wherein the subject has been diagnosed with cancer.

163. The method of claim 162, wherein the cancer comprises a cancer that is positive for expression of the peptide.

164. The method of any one of claims 156-163, wherein the cancer comprises leukemia, lung cancer, or skin cancer.

165. The method of any one of claims 156-164, wherein the cancer comprises a solid tumor.

166. The method of any one of claims 156-165, wherein the cancer comprises lung cancer.

167. The method of claim 166, wherein the lung cancer comprises adenocarcinoma or squamous cell carcinoma.

168. The method of any one of claims 156-167, wherein treating comprises one or more of reducing tumor size; increasing the overall survival rate; reducing the risk of recurrence of the cancer; reducing the risk of progression; and/or increasing the chance of progression-free survival, relapse-free survival, and/or recurrence-free survival.

169. A method of cloning a peptide-specific T cell receptor (TCR), the method comprising
(a) obtaining a starting population of immune effector cells;
(b) contacting the starting population of immune effector cells with the peptide of any one of claims 1-10, thereby generating peptide-specific immune effector cells;
(c) purifying immune effector cells specific to the peptide, and
(d) isolating a TCR sequence from the purified immune effector cells.

170. The method of claim 169, wherein contacting is further defined as co-culturing the starting population of immune effector cells with antigen presenting cells (APCs), artificial antigen presenting cells (aAPCs), or an artificial antigen presenting surface (aAPSs); wherein the APCs, aAPCs, or the aAPSs present the peptide on their surface.

171. The method of claim 170, wherein the APCs are dendritic cells.

172. The method of claim 169, wherein the immune effector cells are T cells, peripheral blood lymphocytes, NK cells, invariant NK cells, NKT cells.

173. The method of claim 169, wherein the immune effector cells have been differentiated from mesenchymal stem cell (MSC) or induced pluripotent stem (iPS) cells.

174. The method of claim 172, wherein the T cells are CD8⁺ T cells, CD4⁺ T cells, CD4⁺ Treg cells, or γδ T cells.

175. The method of claim 172, wherein the T cells are cytotoxic T lymphocytes (CTLs).

176. The method of any one of claims 169-176, wherein obtaining comprises isolating the starting population of immune effector cells from peripheral blood mononuclear cells (PBMCs).

177. The method of any of claims 169-176, wherein the starting population of immune effector cells is obtained from a subject.

178. The method of claim 177, wherein the subject is a human.

179. The method of claim 178, wherein the subject has cancer.

180. The method of any one of claims 169-179, wherein the cancer comprises leukemia, lung cancer, or skin cancer.

181. The method of any one of claims 169-180, wherein the cancer comprises a solid tumor.

182. The method of any one of claims 169-181, wherein the cancer comprises lung cancer.

183. The method of claim 182, wherein the lung cancer comprises adenocarcinoma or squamous cell carcinoma.

184. The method of any one of claims 171-182, wherein the method further comprises introducing the peptide or a nucleic acid encoding the peptide into the dendritic cells prior to the co-culturing.

185. The method of claim 184, where the peptide or nucleic acid encoding the peptide are introduced by electroporation.

186. The method of claim 184, wherein the peptide or nucleic acid encoding the peptide are introduced by adding the peptide or nucleic acid encoding the peptide to the media of the dendritic cells.

187. The method of claim 184, wherein the immune effector cells are co-cultured with a second population of dendritic cells into which the peptide or a nucleic acid encoding the peptide has been introduced.

188. The method of claim 184, wherein purifying is defined as purifying a population of CD4- or CD8-positive and peptide MHC tetramer-positive T cells from the immune effector cells following the co-culturing.

189. The method of claim 188, wherein the population of CD4- or CD8-positive and peptide MHC tetramer-positive T cells are purified by fluorescence activated cell sorting (FACS).

190. The method of claim 189, wherein purifying further comprises generation of a clonal population of peptide-specific immune effector cells by limiting or serial dilution of sorted cells followed by expansion of individual clones by a rapid expansion protocol.

191. The method of claim 190, wherein isolating is defined as cloning of a T cell receptor (TCR) from the clonal population of peptide-specific immune effector cells.

192. The method of any one of claims 169-191, wherein the method further comprises sequencing the TCR alpha and/or beta gene(s) and/or performing grouping of lymphocyte interactions by paratope hotspots (GLIPH) analysis.

193. The method of claim 191 or 192, wherein cloning of the TCR is cloning of a TCR alpha and a beta chain.

194. The method of claim 193, wherein the TCR alpha and beta chains are cloned using a 5′-Rapid amplification of cDNA ends (RACE) method.

195. The method of claim 194, wherein the cloned TCR is subcloned into an expression vector.

196. The method of claim 195, wherein the expression vector comprises a linker domain between the TCR alpha sequence and TCR beta sequence.

197. The method of claim 196, wherein the linker domain comprises a sequence encoding one or more peptide cleavage sites.

198. The method of claim 197, wherein the one or more cleavage sites are a Furin cleavage site and/or a P2A cleavage site.

199. The method of claim 198, wherein the TCR alpha sequence and TCR beta sequence are linked by an IRES sequence.

200. The method of any of claims 195-199, wherein the expression vector is a retroviral or lentiviral vector.

201. The method of claim 200, where a host cell is transduced with the expression vector to generate an engineered cell that expresses the TCR alpha and beta chains.

202. The method of claim 201, wherein the host cell is an immune cell.

203. A TCR produced by the method of any one of claims 169-202.

204. A fusion protein comprising the peptide binding region of the TCR of claim 203 fused to a T cell binding region.

205. The fusion protein of claim 204, wherein the peptide binding region of the TCR comprises the alpha and beta variable regions of the TCR.

206. The fusion protein of claim 204 or 205, wherein the T cell binding region comprises a CD3 binding region.

207. The fusion protein of claim 206, wherein the CD3 binding region comprises an anti-CD3 Fab.

208. A method for prognosing a patient or for detecting T cell responses in a patient, the method comprising: contacting a biological sample from the patient with the peptide of any one of claims 1-10 or the molecular complex of claim 11.

209. The method of claim 208, wherein the biological sample comprises a blood sample or a fraction thereof.

210. The method of claim 209, wherein the biological sample comprises lymphocytes.

211. The method of claim 210, wherein the biological sample comprises a fractionated sample comprising lymphocytes.

212. The method of any one of claims 208-211, wherein the peptide is linked to a solid support.

213. The method of claim 212, wherein the peptide is conjugated to the solid support or is bound to an antibody that is conjugated to the solid support.

214. The method of claim 212, wherein the solid support comprises a microplate, a bead, a glass surface, a slide, or a cell culture dish.

215. The method of any one of claims 208-214, wherein detecting T cell responses comprises detecting the binding of the peptide to the T cell or TCR.

216. The method of any one of claims 208-215, wherein detecting T cell responses comprises an ELISA, ELISPOT, or a tetramer assay.

217. A composition comprising at least one MHC polypeptide and the peptide of any one of claims 1-10.

218. The composition of claim 217, wherein the MHC polypeptide is and/or peptide is conjugated to a detection tag.

219. The composition of claim 217 or 218, wherein the MHC polypeptide and peptide are operatively linked to form a peptide-MHC complex.

220. The composition of claim 219, wherein the MHC polypeptide and peptide are operatively linked through a peptide bond.

221. The composition of claim 219, wherein the MHC polypeptide and peptide are operatively linked through van der Waals forces.

222. The composition of any one of claims 219-221, wherein at least two peptide-MHC complexes are operatively linked to each other.

223. The composition of claim 222, wherein at least 3 or 4 peptide-MHC complexes are operatively linked to each other.

224. The composition of any one of claims 217-223, wherein the average ratio of MHC polypeptides to peptides is 1:1 to 4:1.

225. A method comprising contacting the composition of any one of claims 218-224 with a composition comprising T cells and detecting T cells with bound peptide and/or MHC polypeptide by detecting a detection tag.

226. The method of claim 225, wherein the method further comprises counting the number of T cells bound with peptide and/or MHC.

227. The method of claim 225 or 226, wherein the composition comprising T cells is isolated from a patient having or suspected of having a cancer.

228. The method of claim 227, wherein the cancer comprises a peptide-specific cancer.

229. The method of claim 227, wherein the peptide is selected from a peptide of SEQ ID NO:1 or 2.

230. The method of any one of claims 225-229, wherein the method further comprises sorting the number of T cells bound with peptide and/or MHC.

231. The method of claim 230, wherein the method further comprises sequencing one or more TCR genes from T cells bound with peptide and/or MHC.

232. The method of claim 231, wherein the method further comprises grouping of lymphocyte interactions by paratope hotspots (GLIPH) analysis.

233. A kit comprising the peptide of any one of claims 1-10 in a container.

234. The kit of claim 233, wherein the peptide is comprised in a pharmaceutical preparation.

235. The kit of claim 234, wherein the pharmaceutical preparation is formulated for parenteral administration or inhalation.

236. The kit of claim 233, wherein the peptide is comprised in a cell culture media.
